SEQUENCE LISTING (13 sequences)

SEQ ID NO: 1 - protein, 4 aa, Artificial
  budding mediating motif core sequence
PTAP

SEQ ID NO: 2 - protein, 4 aa, Artificial
  budding mediating motif core sequence
PPXY

SEQ ID NO: 3 - protein, 4 aa, Artificial
  budding mediating motif core sequence
YXXL

SEQ ID NO: 4 - protein, 4 aa, Artificial
  budding mediating motif core sequence
PSAP

SEQ ID NO: 5 - protein, 4 aa, Artificial
  budding mediating motif core sequence
YPDL

SEQ ID NO: 6 - DNA, 156 bp, Human immunodeficiency virus type 1
  CDS (1)..(144)
ctgcagaacagacctgagcccaccgccccacctgctgagagcttccggttcgaagagaccacacccgcccccaagcaggagagcaaggacagagaagcactgaccagcctgaagagcctgttcggcagcgatcccctgagccagtgaggatccgaa

SEQ ID NO: 7 - protein, 48 aa, Human immunodeficiency virus type 1
LQNRPEPTAPPAESFRFEETTPAPKQESKDREALTSLKSLFGSDPLSQ

SEQ ID NO: 8 - DNA, 66 bp, Human immunodeficiency virus type 1
  CDS (1)..(63)
ctgcagaacagacctgagccagtgaggatccgaattcctgcagcccgggggatccgcccgggctag

SEQ ID NO: 9 - protein, 21 aa, Human immunodeficiency virus type 1
LQNRPEPVRIRIPAARGIRPG

SEQ ID NO: 10 - DNA, 1485 bp, Human immunodeficiency virus type 1
atgggagccagagccagcatcctgagaggaggcaaactggacaagtgggagaagattagactgcggcctggaggcaagaaacggtacatgatcaagcacctggtgtgggccagcagagagctggagcggttcgcactgaatcctggcctcctggagaccagcgaaggatgcaaacagatcatgaagcagctccaaccagctctgcagaccggcactgaggaactgagaagcctgtacaacaccgtggccaccctgtactgcgtgcacgagggcgtggaagtgcgggacaccaaggaggccctggaccggatcgaggaagagcagaacaagatccagcaaaagatccagcagaagacccaacaggccgctgatggaaaggtgagccagaactaccccatcgtccagaacctccagggccagatggtgcaccagaagctgagccctcggacactgaacgcctgggtcaaggtgatcgaagagaaggccttcagccctgaagtgatccccatgttcacagctctgagcgaaggcgccactcctcaggacctgaacaccatgctgaacaccgtgggaggccaccaagctgcaatgcagatgctgaaggacaccatcaacgaggaagctgccgagtgggacagactgcatccagtccacgccggacccatcgctcctggccagatgcgggaacctagaggaagcgatatcgctggcactacctccaccctgcaagagcagatcgcttggatgaccagcaacccccctatccccgtcggcgacatctacaagcggtggatcatcctgggcctgaacaagatcgtgagaatgtacagccccgtgagcatcctggacatcaagcaaggacctaaggagcccttcagagactacgtcgaccggttctttaagactctgagagccgagcaggcaacccaggaggtgaagaactggatgaccgacacactgctggtccagaacgccaaccccgactgcaagaccatcctgaaggctctgggacccggcgccacactggaagagatgatgacagcatgccagggcgtcggaggaccaagccacaaagcaagagtgctcgccgaggccatgagccagaccaacagcgtgaatatcctgatgcagaagagcaacttcaaaggcaacaagcggatggtcaagtgcttcaactgtggcaaggaaggacacatcgcacggaactgcagagctccacggaagaaaggctgctggaagtgcggcaaggaaggacaccagatgaaggactgcacagagcggcaagcaaacttcctcggaaagatctggccaagccacaagggaagacccggcaatttcctgcagaacagacctgagcccaccgccccacctgctgagagcttccggttcgaagagaccacacccgcccccaagcaggagagcaaggacagagaagcactgaccagcctgaagagcctgttcggcagcgatcccctgagccagtga

SEQ ID NO: 11 - DNA, 38 bp, Artificial
  mutation primer
tcctgcagaacagacctgagcctgctgagagcttccgg

SEQ ID NO: 12 - DNA, 38 bp, Artificial
  mutation primer
ccggaagctctcagcaggctcaggtctgttctgcagga

SEQ ID NO: 13 - DNA, 12 bp, Human immunodeficiency virus type 1
cccaccgcccca

(19) United States
(12) Patent Application Publication
(10) Pub. No.: US 2005/0220816 A1
(43) Pub. Date: Oct. 6, 2005

(54) Mutant viral nucleic acids and vaccine containing same

(75) Inventors: Anthony D. Cristillo, Silver Spring, MD (US); Vaniambadi Kalyanaraman, Rockville, MD (US); Ranajit Pal, Gaithersburg, MD (US)

Correspondence Address: Samir R. Patel, Esq.;bioMerieux, Inc., Patent Department, 100 Rodolphe Street, Durham, NC, 27712, US

(73) Assignee: Advanced BioScience Laboratories, Inc.

(21) Appl. No.: 10/814,689

(22) Filed: Mar. 31, 2004

(51) Int. Cl. C12Q 1/70; A61K 39/12; A61K 39/21; C07K 14/16; C07H 21/02

(52) U.S. Cl. 424/208.1; 435/456; 536/23.72; 530/350

(57) ABSTRACT

The present invention is directed to a nucleic acid comprising a deletion mutant of a PTAP (SEQ ID NO:1) motif and/or PPXY (SEQ ID NO:2) and/or YXXL (SEQ ID NO:3) motif in the late or L domain of a viral protein, where the L domain mediates the budding process. Examples of such viral proteins containing L domains are the retroviral gag proteins and the matrix proteins of rhabdoviruses and filoviruses. In addition, the present invention is directed to a vector containing (a) this nucleic acid or (b) this nucleic acid and one or more nucleic acids encoding other structural and regulatory viral proteins. The present invention is further directed to vaccines containing the nucleic acid or vector for the purpose of augmenting a cellular immune response.

### BACKGROUND OF THE INVENTION

The present invention is directed to a nucleic acid comprising a deletion mutant of a PTAP (SEQ ID NO:1) motif and/or PPXY (SEQ ID NO:2) and/or YXXL (SEQ ID NO:3) motif in the late or L domain of a viral protein, where the L domain mediates the budding process. Examples of such viral proteins containing L domains are the retroviral gag proteins and the matrix proteins of rhabdoviruses and filoviruses. In addition, the present invention is directed to a vector containing (a) this nucleic acid or (b) this nucleic acid and one or more nucleic acids encoding other structural and regulatory viral proteins. The present invention is further directed to vaccines containing the nucleic acid or vector for the purpose of augmenting a cellular immune response.

The publications and other materials used herein to illuminate the background of the invention, and in particular, cases to provide additional details respecting the practice, are incorporated by reference, and for convenience are referenced in the following text and are listed in the appended bibliography.

While neutralizing antibodies are critical components of anti-HIV immunity, there is also increasing evidence that emphasize the importance of cytotoxic T lymphocytes as necessary effectors in controlling HIV-1 (1-7). To date, one major obstacle that has been encountered in HIV-1 vaccine development has been the generation of a vaccine that elicits both humoral immune and cell mediated immune (CMI) responses. While some investigators have attempted to augment the CMI response by utilizing a DNA prime plus protein boost methodology (8,9), others have used DNA prime with a modified virus Ankara (MVA; an attenuated vaccinia virus) (10-12), or DNA vaccines in conjunction with CD4+ Th1 promoting cytokines (8,9) or with other novel adjuvants (CRL8623) (13). These approaches to increase the CMI responses elicited by an HIV-1 vaccine have produced both promising and varied results. Several formulations are currently being further evaluated using different clades of HIV-1 in murine and nonhuman primate model systems. New exploratory studies are therefore needed to continue testing innovative approaches to augment the CMI response in order to help control HIV-1.

Ubiquitination is a post-translational modification of proteins that involves the covalent binding of a 76 amino acid polypeptide, ubiquitin, to a target protein. This multistep process is mediated by the proteins, E1, E2 and E3, that flag protein substrates destined for rapid proteosome-driven degradation. The ubiquitin activating enzyme, E1, is responsible for ATP-dependent ubiquitin activation. Once activated, ubiquitin is transferred to the downstream carrier protein, E2, and then to the isopeptide ligase, E3, that mediates the transfer to a substrate protein. Ubiquitination is involved in the downregulation of membrane receptors, transporters and channels as well as in cell cycle control. Within the immune system, ubiquitination is involved in transcriptional and translational activation, protein kinase activation, and apoptosis. Studies have uncovered that viral particles from HIV-1, SIV and moloney murine leukemia virus (Mo-MuLV) contain an enrichment of unconjugated ubiquitin compared to that found in the cytoplasm (14,15). It has been further noted that Gag proteins of several retroviruses including HIV-1, SIV, Mo-MuLV and Rous sarcoma virus (RSV) are monoubiquitinated and that ubiquitination plays a critical role in late stage processing and budding (14,16,17). To this end, depletion of intracellular free ubiquitin pools, using proteosome inhibitors, has been shown to inhibit viral budding of HIV-1, HIV-2 and RSV. The mechanism by which this ubiquitination occurs and the signaling events that influence budding and viral pathogenesis are not completely understood.

In recent years, deletion analysis has been used to identify regions of viral Gag proteins that are critical for budding. While much of HIV-1 and other retroviral Gag proteins appear to be dispensable for budding, an essential region has been identified to include a particular sequence termed the late assembly domain (18-22). The core element in the late assembly domain has been shown to include the following conserved sequences that are functionally interchangeable: PTAP, PPxY and YPDL (19-21,23,24). This core element has been found to be required for efficient pinching off of the virus bud (18,20,21). In addition, expression of HIV-1, RSV or BLV Gag late domain deletion mutants results in host cells covered with viral particles that remain tethered to the membrane (16,17). The late domain core sequences (PTAP (SEQ ID NO:1), PPXY (SEQ ID NO:2), PSAP (SEQ ID NO:3), and YXXL (SEQ ID NO:4)) are well conserved throughout the retroviridae family (PTAP (SEQ ID NO:1) for HIV-1, SIV, HTLV-II; PPXY (SEQ ID NO:2; X is any amino acid) for RSV, Mo-MuLV, BLV, HTLV-I, HTLV-II; PSAP (SEQ ID NO:4) for HIV-2, FIV, BIV, CAEV, Visna Virus; and is YXXL (SEQ ID NO:3; X is any amino acid), such as YPDL (SEQ ID NO:5) for EIAV); a finding that is consistent with its importance in viral pathogenesis.

Ongoing studies seeking to bridge the gap “mechanistically” between viral budding, ubiquitination and the late assembly domain core element have delineated that the tumor susceptibility gene product (Tsg101), a key molecule in vacuolar protein sorting (vps) and the E3 ligase, Nedd4, bind to PTAP (SEQ ID NO:1) and PPXY (SEQ ID NO:2) sequences, respectively, and are integral components of late stage viral budding (25,26). While current studies investigate cellular factors of the vps and ubiquitin pathways that bind Tsg101 and Nedd4 (27) and examine their effects on viral budding, less attention is being paid to exploiting the PTAP (SEQ ID NO:1) motif in HIV-1 vaccine development.

Thus, there is a need to develop safe and effective prophylactic vaccines against retroviruses, including human immunodeficiency virus (HIV), rhabdoviruses and filoviruses that elicit both robust humoral and CMI responses. Currently, such a vaccine for HIV does not exist. The health implications and urgency for such a vaccine are obvious given that more than 40 million people are living with HIV and that currently AIDS is a leading cause of death worldwide among infectious diseases.

### SUMMARY OF THE INVENTION

The present invention is directed to a nucleic acid comprising a deletion mutant of a PTAP (SEQ ID NO:1) motif and/or PPXY (SEQ ID NO:2) and/or YXXL (SEQ ID NO:3) motif in the late or L domain of a viral protein, where the L domain mediates the budding process. Examples of such viral proteins containing L domains include the retroviral gag proteins, the matrix proteins of rhabdoviruses and filoviruses (82) and proteins associated with budding in other viruses such as the viruses with the budding mediating motif set forth in Table 1. In addition, the present invention is directed to a vector containing (a) this nucleic acid or (b) this nucleic acid and one or more nucleic acids encoding other structural and regulatory viral proteins. The present invention is further directed to vaccines containing the nucleic acid or a vector for the purpose of augmenting a cellular immune response.

In a first aspect, the present invention provides a nucleic acid comprising a deletion mutant of the budding mediating motif. Such a deletion mutant is with respect to the “PTAP” (SEQ ID NO:1) motif, and/or the “PPXY” (SEQ ID NO:2) motif and/or the “YXXL” (SEQ ID NO:3) motif in the late domain of a viral protein that is associated with budding, such as a Gag protein of retroviruses, a matrix protein of rhabdoviruses and filoviruses or proteins associated with budding in other viruses such as the viruses with the budding mediating motif set forth in Table 1. In one embodiment, the nucleic acid comprises a deletion mutant of the human immunodeficiency virus 1 (HIV-1) Gag p6 “PTAP” (SEQ ID NO:1) motif. In a second embodiment, the nucleic acid comprises a deletion mutant of the simian immunodeficiency virus (SIV) Gag “PTAP” (SEQ ID NO:1) motif.

In a second aspect, the present invention provides a vector, e.g., a plasmid, comprising a nucleic acid which comprises a deletion mutant of the budding mediating motif described herein. In one embodiment, the vector comprises a nucleic acid comprising a deletion mutant of the human immunodeficiency virus 1 (HIV-1) Gag p6 “PTAP” (SEQ ID NO:1) motif. In a second embodiment, the vector comprises a nucleic acid comprising a deletion mutant of the simian immunodeficiency virus (SIV) Gag “PTAP” (SEQ ID NO:1) motif.

In a third aspect, the present invention provides a vector, e.g., a plasmid, comprising a nucleic acid which comprises a deletion mutant of the budding mediating motif described herein and one or more nucleic acids, each of which encodes an additional structural or regulatory viral protein, such as Pol, Env, Rev, Tat or Nef. In one embodiment directed to HIV-1, the vector comprises a nucleic acid comprising a deletion mutant of the human immunodeficiency virus 1 (HIV-1) Gag p6 “PTAP” (SEQ ID NO:1) motif and a nucleic acid encoding one of HIV-1 Pol, HIV-1 Env, HIV-1 Rev, HIV-1 Tat or HIV-1 Nef. In a second embodiment directed to HIV-1, the vector comprises a nucleic acid comprising a deletion mutant of the human immunodeficiency virus 1 (HIV-1) Gag p6 “PTAP” (SEQ ID NO:1) motif and two nucleic acids encoding two of HIV-1 Pol, HIV-1 Env, HIV-1 Rev, HIV-1 Tat or HIV-1 Nef. In a third embodiment directed to HIV-1, the vector comprises a nucleic acid comprising a deletion mutant of the human immunodeficiency virus 1 (HIV-1) Gag p6 “PTAP” (SEQ ID NO:1) motif and three nucleic acids encoding three of HIV-1 Pol, HIV-1 Env, HIV-1 Rev, HIV-1 Tat or HIV-1 Nef. In a fourth embodiment directed to HIV-1, the vector comprises a nucleic acid comprising a deletion mutant of the human immunodeficiency virus 1 (HIV-1) Gag p6 “PTAP” (SEQ ID NO:1) motif and four nucleic acids encoding four of HIV-1 Pol, HIV-1 Env, HIV-1 Rev, HIV-1 Tat or HIV-1 Nef. In a fifth embodiment directed to HIV-1, the vector comprises a nucleic acid comprising a deletion mutant of the human immunodeficiency virus 1 (HIV-1) Gag p6 “PTAP” (SEQ ID NO:1) motif and five nucleic acids encoding all of the following HIV-1 proteins: Pol, HIV-1 Env, HIV-1 Rev, HIV-1 Tat or HIV-1 Nef. In a first embodiment directed to SIV, the vector comprises a nucleic acid comprising a deletion mutant of the simian immunodeficiency virus (SIV) Gag “PTAP” (SEQ ID NO:1) motif in SIV Gag and a nucleic acid encoding one of SIV Pol, SIV Env, SIV Rev, SIV Tat or SIV Nef. In a second embodiment directed to SIV, the vector comprises a nucleic acid comprising a deletion mutant of the simian immunodeficiency virus (SIV) Gag p6 “PTAP” (SEQ ID NO:1) motif and two nucleic acids encoding two of SIV Pol, SIV Env, SIV Rev, SIV Tat or SIV Nef. In a third embodiment directed to SIV, the vector comprises a nucleic acid comprising a deletion mutant of the simian immunodeficiency virus (SIV) Gag p6 “PTAP” (SEQ ID NO:1) motif and three nucleic acids encoding three of SIV Pol, SIV Env, SIV Rev, SIV Tat or SIV Nef. In a fourth embodiment directed to SIV, the vector comprises a nucleic acid comprising a deletion mutant of the simian immunodeficiency virus (SIV) Gag p6 “PTAP” (SEQ ID NO:1) motif and four nucleic acids encoding four of SIV Pol, SIV Env, SIV Rev, SIV Tat or SIV Nef. In a fifth embodiment directed to SIV, the vector comprises a nucleic acid comprising a deletion mutant of the simian immunodeficiency virus (SIV) Gag p6 “PTAP” (SEQ ID NO:1) motif and five nucleic acids encoding all of the following SIV proteins: Pol, SIV Env, SIV Rev, SIV Tat or SIV Nef.

In a fourth aspect, the present invention provides a nucleic acid comprising a molecular clone of a virus with a deletion mutant of the budding mediating motif and to a vector containing such a nucleic acid. In one embodiment, the nucleic acid comprises a molecular clone of HIV-1 with a deletion in the Gag “PTAP” (SEQ ID NO:1) motif and to a vector, e.g., a plasmid, containing such a nucleic acid. In a second embodiment, the nucleic acid comprises a molecular clone of SIV with a deletion in the Gag “PTAP” (SEQ ID NO:1) motif and to a vector, e.g., a plasmid, containing such a nucleic acid.

In a fifth aspect, the present invention provides products produced by these nucleic acids, e.g., mRNA, protein and viral particles.

In a sixth aspect, the present invention provides compositions comprising these nucleic acids and/or their expression products.

In a seventh aspect, the present invention provides host cells comprising these nucleic acids, their expression products, vectors or virus-like particles.

In an eighth aspect, the present invention provides a vaccine comprising the above described nucleic acids or vectors.

In a ninth aspect, the present invention provides a method for immunization by administering an effective amount of the vaccine provided by the invention.

In a tenth aspect, the present invention provides a method for augmenting a cellular immune response to an immunodeficiency virus by administering an effective amount of the vaccine provided by the present invention.

### BRIEF DESCRIPTION OF THE FIGURES

FIG. 1 shows the HIV-1 Gag p6 region and the p6 deletion mutant. FIG. 1 illustrates the nucleotide sequence (SEQ ID NO:6) and amino acid sequence (SEQ ID NO:7) of the HIV-1 Gag p6 region that contains the PTAP (SEQ ID NO:1) motif. It also indicates the p6 region (box) that was deleted using Bpu 101 to generate the p6 mutant having the nucleotide sequence (SEQ ID NO:8) and amino acid sequence (SEQ ID NO:9).

FIGS. 2A-2D show increased IFNγ production in CD8+ and CD4+ T cells elicited by HIV-1 p6 Mutant Gag vaccine. Five to seven week old Balb/c mice were injected intramuscularly with 50 μg of either HIV-1 Gag (GagA7) or p6 mutant Gag. Some of the mice (FIG. 2D) also received a Gag p42 protein boost administered intramuscularly. Five mice per test group received one injection every 2 weeks for a total of 3 DNA injections. Two weeks after the final injection, mice were sacrificed and spleens were harvested. Mice in the DNA plus protein vaccine group were given 30 ug Gag p42 protein at weeks 5 and 7 and splenocytes were removed at week 9. Splenocytes were prepared and stimulated for 6 hours with either Gag peptide pool (1 μg/ml) or PMA (10 ng/ml) plus lonomycin (1.6 μM). Following stimulation, cell surface staining was carried out using rat anti-mouse FITC-conjugated anti-CD3, PerCP-conjugated anti-CD4 and APC-conjugated anti-CD8 monoclonal antibodies (BD Biosciences). Intracellular cytokine staining was carried out using rat anti-mouse PE-conjugates IFNγ, TNFα and Th2 (IL4, IL5, IL10) monoclonal antibodies followed by cytometry analysis. FIGS. 2A and 2B illustrate dot blot analyses (1 representative animal from the DNA only immunized groups) of CD8+ T cells producing IFNγ (FIG. 2A, upper) and TNFα (FIG. 2A, lower) and of CD4+ T cells producing IFNγ (FIG. 2B, upper) and TNFα (FIG. 2B, lower). A summary of the results from one preliminary study showing the mean levels of Th1 and Th2 cytokines relative to unstimulated controls is graphically presented for the DNA (FIG. 2C) and DNA plus protein (FIG. 2D) vaccinated mice.

FIGS. 3A-3B show that levels of CD8+ and CD4+ T cell proliferation elicited by HIV-1 p6 Mutant Gag and GagA7 DNA vaccines are comparable. This was also found in mice vaccinated with a DNA prime plus protein boost strategy (data not shown). Vaccination conditions, tissue harvesting and splenocyte preparation were carried out as outlined in FIGS. 2A-2C. Splenocytes were prepared and labeled with CFSE (1 μM) for 15 min at 37 C. Labeled cells were stimulated with either Gag peptide pool (1 μg/ml) or PMA (2.5 ng/ml) plus Ionomycin (1.5 μM) for 72 hours. Following 72 hours, cells were stained with rat anti-mouse PE-conjugated anti-CD3, PerCP-conjugated anti-CD4 and APC-conjugated anti-CD8 monoclonal antibodies (BD Biosciences) and analysed by cytometry (FACScalibur, BD BioSciences). FIGS. 3A and 3B illustrate a density blot analyses of CD8+ (FIG. 3A) and CD4+ (FIG. 3B) T cells from representative animals of the 2 test groups.

FIG. 4 shows increased CTL activity elicited by HIV-1 P6 Mutant Gag vaccine in the absence (FIG. 4A) and presence (FIG. 4B) of a Gag p42 protein boost. Vaccination conditions, tissue harvesting and splenocyte preparation were carried out as outlined in FIGS. 2A-2D. Splenocytes were cultured in the presence of Gag peptide pool (1 μg/ml) for 8 days. Complete media was supplemented with recombinant IL2 (28 units/ml). Following stimulation, various concentrations of these effector cells were co-cultured for 2 hr with P815 target cells (previously labeled with Cell Tracker Orange (3 μM) and Gag peptide pool (1 μg/ml) for 1 hr). Following co-culture with P815 cells, cells were cultured in the presence of PhiPhiLux substrate (Oncolmmunin, Gaithersburg, Md.) for 1 hr and then analyzed by cytometry (FACScalibur, BD BioSciences).

FIG. 5 shows an example of p6 mutation primers (SEQ ID NO:11 and SEQ ID NO:12).

### DETAILED DESCRIPTION OF THE INVENTION

A long-term objective in the development of vaccines, particularly human immunodeficiency virus (HIV) vaccines, is to develop a safe and effective vaccine that elicits both robust humoral and cell mediated immune (CMI) responses. In accordance with this objective, one approach to improve HIV vaccine formulations and the development of a successful vaccine is to focus on augmenting the cellular immune response. The present invention provides a novel means to accomplish this goal through the use of a DNA vaccine that expresses a mutated Gag protein with a processing and budding defect. The mutated Gag protein and virus-like particles that result from the expression of mutant Gag in host cells will be defective in late stage processing and in their ability to bud. They thus have a prolonged intracellular presence, which give the host cells' machinery increased time to degrade and process the viral protein for MHC class I conjugation and surface presentation, yielding greater cytotoxic T lymphocyte (CTL) activity. Thus, the increased intracellular retention of the mutant Gag protein and/or virus-like particles elicit an enhanced CMI response due to disrupted late stage processing and budding. The present invention also provides a novel means to accomplish the similar goal for other retroviruses and other viruses such as rhabdoviruses and filoviruses through the use of a mutated gag protein for the retroviruses, a mutated matrix protein for the rhabdoviruses and filoviruses and a mutated protein associated with budding for other viruses listed in Table 1 which contain the budding mediating motif.

In addition to the DNA vaccine expressing the mutated Gag protein (for retroviruses), mutated matrix protein (for rhabdoviruses and filoviruses) or other mutated viral protein with the mutated budding mediating motif, the DNA vaccine can also include DNAs encoding other structural and regulatory viral proteins (e.g., Pol, Env, Rev, Tat and Nef). There is also a greater intracellular retention of these viral components, in addition to the mutated Gag protein (for retroviruses), mutated matrix protein (for rhabdoviruses and filoviruses) or other mutated viral protein with the mutated budding mediating motif and resultant virus-like particles, so as to facilitate MHC class I conjugation, surface presentation and CTL activity directed against varied epitopes.

Further, administration of this enhanced, mutant vaccine as a DNA prime plus protein boost strategy or other (i.e., live or attenuated virus) boost approach augments the CTL activity further by stimulating the necessary CD4+ T helper cell response.

For convenience, certain terms employed in the specification, examples, and appended claims are collected here.

As used in this specification and the appended claims, the singular forms “a,” “an” and “the” include plural references unless the content clearly dictates otherwise. Thus, for example, reference to “an antigen” includes a mixture of two or more such agents.

The term “budding mediating motif” as used herein refers to the amino acid sequence PTAP (SEQ ID NO:1), PPXY (SEQID NO:2) YXXL (SEQ ID NO:3) or a combination of any of these three amino acid sequences which are found in L domains of viral proteins and which mediate the budding process.

The term “PTAP (SEQ ID NO:1) motif” as used herein refers to the amino acid sequence PTAP (SEQ ID NO:1) found in a late or L domain of a viral protein which mediates the budding process. Examples of such viral proteins containing L domains include the retroviral gag proteins and the matrix proteins of rhabdoviruses and filoviruses (82).

The term “PPXY (SEQ ID NO:2) motif” as used herein refers to the amino acid sequence PPXY (SEQ ID NO:2) found in a late or L domain of a viral protein which mediates the budding process. Examples of such viral proteins containing L domains include the retroviral gag proteins and the matrix proteins of rhabdoviruses and filoviruses (82).

The term “YXXL (SEQ ID NO:3) motif” as used herein refers to the amino acid sequence YXXL (SEQ ID NO:3) found in a late or L domain of a viral protein which mediates the budding process. Examples of such viral proteins containing L domains include the retroviral gag proteins and the matrix proteins of rhabdoviruses and filoviruses (82).

The term “Gag PTAP (SEQ ID NO:1) motif” as used herein refers to the amino acid sequence PTAP (SEQ ID NO:1) found in HIV-1 Gag p6 at amino acid residues (455-458) of the HIV-1 Gag protein sequence (HIV-1 gag DNA (Accession No. NC_{—}001802); HIV-1 Gag Protein (Accession No. NP_{—}057850.1); HIV-1 Gag p6 (Accession No. NP_{—}579883.1) or a codon optimized gag construct, such as the one set forth in SEQ ID NO:11)) or found in SIV Gag at amino acid residues 458-461of the SIV Gag protein (Accession # AAB59905 ) sequence. The term “Gap PTAP motif” also includes modifications by deletion or altering amino acids to other amino acids that will disrupt budding.

The term “deletion mutant of the budding mediated motif” as used herein refers to a mutant viral protein in which the four amino acids of the PTAP (SEQ ID NO:1) motif, and/or PPXY (SEQ ID NO:2) motif and/or YXXL (SEQ ID NO:3) motif have been deleted to produce a viral protein which has a processing and budding defect. The deletion mutant of the budding mediated motif may also include deletions of amino acids surrounding the PTAP (SEQ ID NO:1) motif, and/or PPXY (SEQ ID NO:2) motif and/or YXXL (SEQ ID NO:3) motif in the viral protein, such as a retroviral gag protein or a matrix protein of a rhabdovirus or a filovirus.

The term “deletion mutant of the Gag PTAP (SEQ ID NO:1) motif” as used herein refers to a mutant Gag in which the four amino acids of the PTAP (SEQ ID NO:1) motif have been deleted to produce a Gag protein which has a processing and budding defect. The deletion mutant of the Gag PTAP (SEQ ID NO:1) motif may also include deletions of amino acids surrounding the Gag PTAP (SEQ ID NO:1) motif in the viral protein, such as HIV-1 p6.

The term “processing and budding defect” as used herein refers to a state in which the virus-like-particle that results from the expression of above mutant viral protein, such as a mutant retroviral gag protein or a mutant matrix protein of a rhabdovirus or a filovirus, cannot “pinch off” or bud from the host cell surface and is therefore trapped either at the membrane or intracellularly.

The term “nucleic acid” as used herein refers to any natural and synthetic linear and sequential arrays of nucleotides and nucleosides, for example cDNA, genomic DNA, mRNA, tRNA, oligonucleotides, oligonucleosides and derivatives thereof. For ease of discussion, such nucleic acids may be collectively referred to herein as “constructs,” “plasmids,” or “vectors.” Representative examples of the nucleic acids of the present invention include bacterial plasmid vectors including expression, cloning, cosmid and transformation vectors such as, but not limited to, pBR322, animal viral vectors such as, but not limited to, modified adenovirus, influenza virus, polio virus, pox virus, retrovirus, and the like, vectors derived from bacteriophage nucleic acid, and synthetic oligonucleotides like chemically synthesized DNA or RNA. The term “nucleic acid” further includes modified or derivatized nucleotides and nucleosides such as, but not limited to, halogenated nucleotides such as, but not only, 5-bromouracil, and derivatized nucleotides such as biotin-labeled nucleotides. In addition, the nucleotide sequence of the nucleic acid can be designed to contain the appropriate codons for the particular desired amino acid sequence. In general, one will select preferred codons for the intended host in which the sequence will be expressed.

The term “isolated nucleic acid” as used herein refers to a nucleic acid with a structure (a) not identical to that of any naturally occurring nucleic acid or (b) not identical to that of any fragment of a naturally occurring genomic nucleic acid spanning more than three separate genes, and includes DNA, RNA, or derivatives or variants thereof. The term includes, but is not limited to, the following: (a) a DNA which has the sequence of part of a naturally occurring genomic molecule but is not flanked by at least one of the coding sequences that flank that part of the molecule in the genome of the species in which it naturally occurs; (b) a nucleic acid incorporated into a vector or into the genomic nucleic acid of a prokaryote or eukaryote in a manner such that the resulting molecule is not identical to any vector or naturally occurring genomic DNA; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene, i.e., a gene encoding a fusion protein, and (e) a recombinant nucleotide sequence that is part of a hybrid sequence that is not naturally occurring.

It is advantageous for some purposes that a nucleotide sequence is in purified form. The term “purified” in reference to nucleic acid represents that the sequence has increased purity relative to the natural environment. The tem “substantially purified” general refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Synthetic” sequences, as used herein, refers to polynucleotides, e.g. gag, env, pol, etc., whose expression has been optimized, for example, by codon substitution, deletions, replacements and/or inactivation of inhibitory sequences. “Wild-type” or “native” sequences, as used herein, refers to polypeptide encoding sequences that are essentially as they are found in nature.

As used herein the terms “polypeptide” and “protein” refer to a polymer of amino acids of three or more amino acids in a serial array, linked through peptide bonds. The tenn “polypeptide” includes proteins, protein fragments, protein analogues, oligopeptides and the like. The term “polypeptides” contemplates polypeptides as defined above that are encoded by nucleic acids, produced through recombinant technology, isolated from an appropriate source, or are synthesized. The term “polypeptides” further contemplates polypeptides as defined above that include chemically modified amino acids or amino acids covalently or noncovalently linked to labeling ligands.

The term “gene” or “genes” as used herein refers to nucleic acid sequences (including both RNA or DNA) that encode genetic information for the synthesis of a whole RNA, a whole protein, or any portion of such whole RNA or whole protein. Genes that are not naturally part of a particular organism's genome are referred to as “foreign genes”, “heterologous genes” or “exogenous genes” and genes that are naturally a part of a particular organism's genome are referred to as “endogenous genes”.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein or polypeptide or a portion thereof.

The term “transcription regulatory sequences” as used herein refers to nucleotide sequences that are associated with a gene nucleic acid sequence and which regulate the transcriptional expression of the gene. The “transcription regulatory sequences” may be isolated and incorporated into a vector nucleic acid to enable regulated transcription in appropriate cells of portions of the vector DNA. The “transcription regulatory sequence” may precede, but are not limited to, the region of a nucleic acid sequence that is in the region 5′ of the end of a protein coding sequence that may be transcribed into mRNA. Transcriptional regulatory sequences may also be located within a protein coding region, in regions of a gene that are identified as “intron” regions, or may be in regions of nucleic acid sequence that are in the region of nucleic acid.

The term “coding region” or “coding sequence” or a sequence which “encodes” a selected polypeptide as used herein refers to a continuous linear arrangement of nucleotides that may be translated into a protein. A full length coding region is translated into a full length protein; that is, a complete protein as would be translated in its natural state absent any post-translational modifications. A full length coding region may also include any leader protein sequence or any other region of the protein that may be excised naturally from the translated protein. A coding sequence is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence.

The term “nucleic acid vector” or “vector” as used herein refers to a natural or synthetic single or double stranded plasmid or viral nucleic acid molecule that can be transfected or transformed into cells and replicate independently of, or within, the host cell genome. A circular double stranded plasmid can be linearized by treatment with an appropriate restriction enzyme based on the nucleotide sequence of the plasmid vector. A nucleic acid can be inserted into a vector by cutting the vector with restriction enzymes and ligating the pieces together. The nucleic acid molecule can be RNA or DNA.

The term “expression vector” as used herein refers to a nucleic acid vector that may further include at least one regulatory sequence operably linked to a nucleotide sequence coding for the protein or antigen of interest or a nucleic acid of the present invention. Regulatory sequences are well recognized in the art and may be selected to ensure good expression of the linked nucleotide sequence without undue experimentation by those skilled in the art. As used herein, the term “regulatory sequences” includes promoters, enhancers, and other elements that may control expression. The vector may also include a bacterial and/or mammalian origin of replication, one or more selectable markers, a signal which allows the plasmid construct to exist as single-stranded DNA, a multiple cloning site and the like. Standard molecular biology textbooks such as Sambrook and Russell, eds., Molecular Cloning: A Laboratory Manual, 3rd Ed., Cold Spring Harbor Press (2001) may be consulted to design suitable expression vectors, promoters, and other expression control elements. It should be recognized, however, that the choice of a suitable expression vector depends upon multiple factors including the choice of the host cell to be transformed and/or the type of protein to be expressed.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

The terms “transformation” and “transfection” as used herein refer to the process of inserting a nucleic acid into a host. Many techniques are well known to those skilled in the art to facilitate transformation or transfection of a nucleic acid into a prokaryotic or eukaryotic organism. These methods involve a variety of techniques, such as treating the cells with high concentrations of salt such as, but not only a calcium or magnesium salt, an electric field, detergent, or liposome mediated transfection, to render the host cell competent for the uptake of the nucleic acid molecules.

The term “recombinant cell” refers to a cell that has a new combination of nucleic acid segments that are not covalently linked to each other in nature. A new combination of nucleic acid segments can be introduced into an organism using a wide array of nucleic acid manipulation techniques available to those skilled in the art. A recombinant cell can be a single eukaryotic cell, or a single prokaryotic cell, or a mammalian cell. The recombinant cell can harbor a vector that is extragenomic. An extragenomic nucleic acid vector does not insert into the cell's genome. A recombinant cell can further harbor a vector or a portion thereof that is intragenomic. The term intragenomic defines a nucleic acid construct incorporated within the recombinant cell's genome. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition.

The term “recombinant nucleic acid” as used herein refers to combinations of at least two nucleic acid sequences that are not naturally found in a eukaryotic or prokaryotic cell. The nucleic acid sequences may include, but are not limited to nucleic acid vectors, gene expression regulatory elements, origins of replication, sequences that when expressed confer antibiotic resistance, and protein-encoding sequences. The term “recombinant polypeptide” is meant to include a polypeptide produced by recombinant DNA techniques such that it is distinct from a naturally occurring polypeptide either in its location, purity or structure. Generally, such a recombinant polypeptide will be present in a cell in an amount different from that normally observed in nature.

As used herein, the term “virus-like particle” or “VLP” refers to a nonreplicating, viral shell, derived from any of several viruses discussed further below. VLPs are generally composed of one or more viral proteins, such as, but not limited to those proteins referred to as capsid, coat, shell, surface and/or envelope proteins, or particle-forming polypeptides derived from these proteins, including the proteins described herein. VLPs can form spontaneously upon recombinant expression of the protein in an appropriate expression system. Methods for producing particular VLPs are known in the art and discussed more fully below. The presence of VLPs following recombinant expression of viral proteins can be detected using conventional techniques known in the art, such as by electron microscopy, biophysical characterization, and the like. For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions.

By “subject” or “patient” is meant any member of the subphylum chordata, including, without limitation, humans and other primates, including non-human primates such as chimpanzees and other apes and monkey species. The term does not denote a particular age. Thus, both adult and newborn individuals are intended to be covered. The system described above is intended for use in any of the above vertebrate species, since the immune systems of all of these vertebrates operate similarly.

By “nucleic acid immunization” is meant the introduction of a nucleic acid molecule encoding one or more selected antigens into a host cell, for the in vivo expression of an antigen, antigens, an epitope, or epitopes. The nucleic acid molecule can be introduced directly into a recipient subject, such as by injection, inhalation, oral, intranasal and mucosal administration, or the like, or can be introduced ex vivo, into cells which have been removed from the host. In the latter case, the transformed cells are reintroduced into the subject where an immune response can be mounted against the antigen encoded by the nucleic acid molecule.

The term “immunizing” or “immunization,” as used herein, refers to the production of an immune response in a patient that protects (partially or totally) from the manifestations of infection (i.e., disease) caused by a pathogen. A patient immunized by the present invention will not be infected by the pathogen or will be infected to a lesser extent than would occur without immunization. Immunizations may be either prophylactic or therapeutic in nature. That is, both previously uninfected and infected patients may be immunized with the present invention.

An “immunological response” or “immune response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytotoxic T lymphocytes (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

A composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host.

An “immunogenic composition” or is a composition that comprises an antigenic molecule where administration of the composition to a subject results in the development in the subject of a humoral and/or a cellular immune response to the antigenic molecule of interest.

“Immunity” means partial or complete protection of an organism against diseases caused by an infectious agent due to a successful elimination of a preceding infection with the infectious agent or a characteristic part thereof. Immunity is based on the existence, induction, and activation of specialized cells of the immune system.

The term “DNA transcription unit” as used herein“refers to a polynucleotide sequence that includes at least two components: antigen-encoding DNA and transcriptional promoter elements. A DNA transcription unit may optionally include additional sequences, such as enhancer elements, splicing signals, termination and polyadenylation signals, viral replicons, and/or bacterial plasmid sequences. The DNA transcription unit can be produced by a number of known methods. For example, DNA encoding the desired antigen can be inserted into an expression vector to construct the DNA transcription unit, as described in Sambrook and Russell, eds., Molecular Cloning: A Laboratory Manual, 3rd Ed., Cold Spring Harbor Laboratory Press (2001), the disclosure of which is incorporated by reference in its entirety.

As used herein, the term “adjuvant” means a substance added to a vaccine to increase a vaccine's immunogenicity. The mechanism of how an adjuvant operates is not entirely known. Some adjuvants are believed to enhance the immune response by slowly releasing the antigen, while other adjuvants are strongly immunogenic in their own right and are believed to function synergistically. Known vaccine adjuvants include, but are not limited to, oil and water emulsions (for example, complete Freund's adjuvant and incomplete Freund's adjuvant), Corynebacterium parvum, Bacillus Calmette Guerin, aluminum hydroxide, glucan, dextran sulfate, iron oxide, sodium alginate, Bacto-Adjuvant, certain synthetic polymers such as poly amino acids and co-polymers of amino acids, saponin, “REGRESSIN” (Vetrepharm, Athens, Ga.), “AVRIDINE” (N, N-dioctadecyl-N′,N′-bis(2-hydroxyethyl)-propanediamine), paraffin oil, detoxified mutants of a bacterial ADP-ribosylating toxins, muramyl peptides, cytokines and immunostimulating agents. Adjuvants also encompass genetic adjuvants such as immunomodulatory molecules encoded in a co-inoculated DNA. The co-inoculated DNA can be in the same vaccine construct as the vaccine immunogen or in a separate DNA vector.

As used herein, the term “pharmaceutically acceptable carrier” means a vehicle for containing the vaccine that can be injected into a subject without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

As used herein, “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

The present invention is more particularly described with reference to the human immunodeficiency virus 1 (HIV-1) or the simian immunodeficiency virus (SIV) in order to provide specific details of the invention. It is understood that the present invention applies to all viruses having budding mediated motifs, such as retroviruses, rhabdoviruses and filovirus, and is not limited to the specific examples of retroviruses described herein.

Thus, in accordance with a first aspect of the present invention, a nucleic acid comprising deletion mutant of the budding mediating motif is provided. Such a deletion mutant is with respect to the “PTAP” (SEQ ID NO:1) motif, and/or the “PPXY” (SEQ ID NO:2) motif and/or the “YXXL” (SEQ ID NO:3) motif in the late domain of a viral protein that is associated with budding, such as a Gag protein of retroviruses or a matrix protein of rhabdoviruses and filoviruses. In one embodiment of this first aspect, the nucleic acid comprises a deletion mutant of the human immunodeficiency virus 1 (HIV-1) Gag p6 “PTAP” (SEQ ID NO:1) motif in HIV-1 Gag. In a second embodiment of this first aspect. the nucleic acid comprises a deletion mutant of the simian immunodeficiency virus (SIV) Gag “PTAP” (SEQ ID NO:1) motif in SIV Gag. Examples of viruses with the budding mediating motif as described herein are set forth in Table 1. Although Table 1 lists all viruses in which the defined motifs exist, it is understood that the present invention is useful for those viruses listed in Table 1 which infect mammals, and especially humans.

TABLE 1

| | Family | | | | | PTAP | PPxY | PTAP |
|---|---|---|---|---|---|---|---|---|
| Order | [Subfamily] | Genus | Type Species | PPxY | PTAP | PPxY | PPxY | PTAP |
| Group I: dsDNA Viruses | | | | | | | | |
| Caudovirales | Lipothrixviridae | Lipothrixvirus | Thermoproteus virus 1 | X | | | | |
| | | | Sulfolobus islandicus | | X | | | |
| | | | filamentous virus | | | | | |
| | Fuselloviridae | Fusellovirus | Sulfolobus virus | X | | | | |
| | | | SSV1 | | | | | |
| | Phycodnaviridae | Chlorovirus | Paramecium bursaria | X | X | | | |
| | | | Chlorella virus 1 | | | | | |
| | | Phaeovirus | Ectocarpus | | X | | | |
| | | | siliculosis virus 1 | | | | | |
| | Poxviridae | | | | | | | |
| | [Chordopoxvirinae] | Orthopoxvirus | Vaccinia virus | X | | | | |
| | | Avipoxvirus | Fowlpox virus | X | | | | |
| | | Leporipoxvirus | Myxoma virus | X | | | | |
| | | Suipoxvirus | Swinepox virus | X | | | | |
| | | Molluscipoxvirus | Molluscum | X | | | X | |
| | | | contagiosum virus | | | | | |
| | | Yatapoxvirus | Yaba monkey tumor | X | | | | |
| | | | virus | | | | | |
| | | Unclassified | Heliothis zea virus | X | | | X | |
| | | | Variola major virus | X | | | | |
| | | | Variola minor virus | X | | | | |
| | | | Camelpox virus | X | | | | |
| | | | Ectromelia virus | X | | | | |
| | | | Monkeypox virus | X | | | | |
| | | | Cowpox virus | X | | | | |
| | | | Yaba-like disease | X | | | | |
| | | | virus | | | | | |
| | | | Lumpy skin disease | X | | | | |
| | | | virus | | | | | |
| | | | Shope fibroma virus | X | | | | |
| | Iridoviridae | Iridovirus | Invertebrate | X | | | | |
| | | | iridescent virus 6 | | | | | |
| | | Lymphocystivirus | Infectious Spleen and | X | | | | |
| | | | kidney necrosis virus | | | | | |
| | Polydnaviridae | Ichnovirus | Campoletis sonorensis | | X | | | |
| | | | ichnovirus | | | | | |
| | Herpesviridae | “Ictalurid | Channel catfish virus | X | | | X | |
| | | herpes-like | (CCV) | | | | | |
| | | viruses” | | | | | | |
| | [Alphaherpesvirinae] | Simplexvirus | Human herpesvirus 1 | X | X | X | X | |
| | | | Herpes Simplex virus 1 | X | X | | X | |
| | | | Herpes Simplex virus 2 | X | X | | X | X |
| | | | Cercopithecine | | X | | | |
| | | | herpesvirus 7 | | | | | |
| | | | Cercopithecine | X | X | | | |
| | | | herpesvirus 9 | | | | | |
| | | Varicellovirus | Simian varicella virus | X | | | | |
| | | | pseudorabies virus | X | | | X | |
| | | | Shbv | | X | | | |
| | [Gammaherpesvirinae] | Lymphocryptovirus | Human herpesvirus 4 | X | X | | X | |
| | | Rhadinovirus | Saimiriine | X | X | X | X | X |
| | | | herpesvirus 2 | | | | | |
| | Baculoviridae | Nucleopolyhedrovirus | Autographa californica | X | X | | | X |
| | | | nucleopolyhedrovirus | | | | | |
| | | | Anagrapha falcifera | | X | | | X |
| | | | nuclear polyhedrosis | | | | | |
| | | | virus | | | | | |
| | | | BmNPV | X | X | | | X |
| | | | LdMNPV | X | X | | | |
| | | | LsMNPV | X | | | | |
| | | | CfMNPV | | X | | | |
| | | | OpMNPV | X | | | | |
| | | Granulovirus | Cydia pomonella | X | | | | |
| | | | granulovirus | | | | | |
| | | | XnGV | X | | | | |
| | Nimaviridae | Whispovirus | White spot syndrome | X | X | | | |
| | | | virus 1 | | | | | |
| | Asfarviridae | Asfivirus | African swine fever | X | | | | |
| | | | virus | | | | | |
| Group II: ssDNA Viruses | | | | | | | | |
| | Geminiviridae | Mastrevirus | Maize streak virus | | X | | | |
| | | | Miscanthus streak | X | | | | |
| | | | virus | | | | | |
| | | Curtovirus | Indian cassava mosaic | X | | | | |
| | | | virus | | | | | |
| | | Begomovirus | Pepper huasteco virus | X | | | | |
| | | | Tomato yellow lead | | X | | | |
| | | | curl virus | | | | | |
| | Circoviridae | Anellovirus | TT virus | X | X | | X | |
| | | | TT-like minivirus | X | | | | |
| | | | SEN virus | X | | | | |
| | Parvoviridae | Parvovirus | Murine Minute virus | X | X | X | X | |
| | [Parvovirinae] | | | | | | | |
| | | | Kilham rat virus | X | X | X | | |
| | | | Feline panleukemia | X | | | | |
| | | | virus | | | | | |
| | | | Mink enteritis virus | X | | | | |
| | | | Raccoon parvovirus | X | | | | |
| | [Densovirinae] | Densovirus | BmDNV | X | | | | |
| | | Iteravirus | Bombyx mori densovirus | X | X | X | | |
| Group III: dsRNA Viruses | | | | | | | | |
| | Reoviridae | Orbivirus | Bluetongue virus | X | | | | |
| | | | AHSV-1 | X | X | X | | |
| | | | AHSV-2 | | X | | | |
| | | | AHSV-8 | | X | | | |
| | | | Saint Croix river | X | X | | | |
| | | | virus | | | | | |
| | | | Epizootic hemorrhagic | X | X | | | |
| | | | disease virus | | | | | |
| | | | Chuzan virus | X | X | | | |
| | | | Broadhaven virus | X | X | | | |
| | | | Equine encephalosis | | X | | | |
| | | | virus-1 | | | | | |
| | | | Equine encephalosis | | X | | | |
| | | | virus-7 | | | | | |
| | | Coltivirus | Colorado tick fever | X | X | | | |
| | | | virus | | | | | |
| | | | Eyach virus | X | X | X | | |
| | | Aquareovirus | Grass carp hemorrhagic | X | | | | |
| | | | virus | | | | | |
| | | Cypovirus | BmCPV | X | | | | |
| | | Phytoreovirus | Rice dwarf virus | X | | | | |
| | | Oryzavirus | Rice ragged stunt | X | | | | |
| | | | virus | | | | | |
| | | Seadornavirus | Banna virus | | X | | | |
| | Birnaviridae | Aquabirnavirus | Infectious pancreatic | X | X | | | |
| | | | necrosis virus | | | | | |
| | Chrysoviridae | Chrysovirus | Penicillium chrysogenum | X | | | | |
| | | | virus | | | | | |
| | Partitiviridae | Partitivirus | Rhizoctonia solani | | X | | | |
| | | | virus | | | | | |
| Group IV: Positive-sense ssRNA Viruses | | | | | | | | |
| | Dicistroviridae | Cripavirus | Taura syndrome virus | X | | | | |
| | | Iflavirus | Infectious flacherie | X | | | | |
| | | | virus | | | | | |
| | Picornaviridae | Enterovirus | Swine vesicula disease | X | | | | |
| | | | virus | | | | | |
| | | Aphthovirus | Foot-and-mouth disease | X | X | | | |
| | | | virus | | | | | |
| | | Erbovirus | Equine rhinitis B | X | | | | |
| | | | virus | | | | | |
| | Comoviridae | Nepovirus | Tomato ringspot virus | X | | | | |
| | | | Grapevine fanleaf virus | X | | | | |
| | Potyviridae | Potyvirus | Dasheen mosaic virus | | X | | | |
| | | Macluravirus | Cardamom mosaic virus | | X | | | |
| | Caliciviridae | Norovirus (was | Norwalk virus | X | | | | |
| | | “Norwalk-like | | | | | | |
| | | viruses”) | | | | | | |
| | | | Desert shield virus | X | | | | |
| | | | Southampton virus | X | | | | |
| | | Sapovirus (was | Sapporo virus | X | | | | |
| | | “Sapporo-like | | | | | | |
| | | viruses”) | | | | | | |
| | | “Hepatitis | Hepatitis E virus | X | X | | | |
| | | E-like viruses” | | | | | | |
| | | Allexivirus | Shallot virus X | X | | | | |
| | | | Garlic virus A | X | X | | | |
| | | | Garlic virus C | X | | | | |
| | | | Garlic virus D | X | | | | |
| | | | Garlic virus E | X | | | | |
| | Astroviridae | Astrovirus | Avian nephritis virus | X | | | | |
| | Tetraviridae | Omegatetravirus | Helicoverpa armigera | X | X | | | |
| | | | stunt virus | | | | | |
| | | | Gill associated virus | X | | | | |
| | | | Yellow head virus | X | | | | |
| | Tombusviridae | Machlomovirus | Maize chlorotic | | X | | | |
| | | | mottle virus | | | | | |
| Nidovirales | Coronaviridae | Coronavirus | Infectious bronchitis | X | | | | |
| | | | virus | | | | | |
| | | | SARS | X | | | | |
| | | | Berne virus | X | | | | |
| | Arteriviridae | Arterivirus | Equine arteritis virus | X | X | | | |
| | | | Lactate dehydrogenase- | X | | | | |
| | | | elevating virus | | | | | |
| | | | Porcine reproductive | X | | | | |
| | | | and respiratory | | | | | |
| | | | syndrome vir | | | | | |
| | Togaviridae | Alphavirus | Sindbis virus | | X | | | |
| | | | Barmah forest virus | X | | | | |
| | | | Ross river virus | X | | | | |
| | | | Equine encephalitis | X | | | | |
| | | | virus | | | | | |
| | | | Mayaro virus | | X | | | |
| | | | Aura virus | | X | | | |
| | | Rubivirus | Rubella virus | X | X | X | X | |
| | Flaviviridae | Flavivirus | Dengue virus-type 3 | | X | | | |
| | | | Kunjin virus | X | | | | |
| | | | Tamana bat virus | X | | | | |
| | | Hepacivirus | Hepatitis C virus | X | X | X | X | |
| | | | HGV | X | X | X | | |
| | Closteroviridae | Closterovirus | Citrus tristeza virus | X | X | | | |
| | | Crinivirus | Lettuce infectious | | | | | |
| | | | yellows virus | | | | | |
| | | Ampelovirus | Grapevine leafroll- | X | | | | |
| | | | associated virus 3 | | | | | |
| | | | Grapevine leafroll- | X | | | | |
| | | | associated virus 5 | | | | | |
| | Luteoviridae | Luteovirus | Barley yellow dwarf | | X | | | |
| | | | virus-PAV | | | | | |
| | | Polerovirus | Cucurbit aphid-born | X | | | | |
| | | | yellow virus | | | | | |
| | | Furovirus | Oat golden stripe | | X | | | |
| | | | virus | | | | | |
| | | Pomovirus | Broad Bean necrosis | X | | | | |
| | | | virus | | | | | |
| | | Pecluvirus | Peanut clump virus | X | X | X | | |
| | | Benyvirus | Beet necrotic yellow | X | | | | |
| | | | vein virus | | | | | |
| | | | BSBMV | X | | | | |
| | | Capillovirus | Apple stem grooving | X | | | | |
| | | | virus | | | | | |
| | | Umbravirus | Groundnut rosette virus | X | | | | |
| | | Unassigned | Sugarcane yellow leaf | X | X | | | |
| | | | virus | | | | | |
| | Tymoviridae | Tymovirus | Turnip yellow mosaic | | X | | | |
| | | | virus | | | | | |
| | | Maculavirus | Grapevine fleck virus | | X | | | |
| | | Carlavirus | Potato virus | X | | | | |
| | | | Aconitum latent virus | X | | | | |
| | | | Garlic latent virus | X | | | | |
| | | | Hop mosaic virus | X | | | | |
| | | | Sugarcane striate | X | | | | |
| | | | mosaic virus | | | | | |
| | | Potexvirus | Potato virus X | X | | | | |
| | | | Narcissus mosaic virus | | X | | | |
| | | | Bamboo mosaic virus | X | | | | |
| | | | Cassava common mosaic | X | | | | |
| | | | virus | | | | | |
| | | | Cymbidium mosaic virus | X | | | | |
| | | | Scallion virus X | X | | | | |
| | | | Clover yellow mosaic | | X | | | |
| | | | virus | | | | | |
| | | Foveavirus | Apple stem pitting | X | | | | |
| | | | virus | | | | | |
| | | | Cherry green ring | X | | | | |
| | | | mottle virus | | | | | |
| | | | African oil palm | X | | | | |
| | | | ringspot virus | | | | | |
| | | | Banana mild mosaic | X | | | | |
| | | | virus | | | | | |
| | | | Cherry necrotic rusty | X | | | | |
| | | | mottle virus | | | | | |
| | | | Rupestris stem pitting | X | | | | |
| | | | associated virus-1 | | | | | |
| | | Vitivirus | Grap vine virus A | X | | | | |
| Group V: Negative-sense ssRNA Viruses | | | | | | | | |
| | Paramyxoviridae | | | | | | | |
| Mononegavirales | [Paramyxovirinae] | Morbillivirus | Measles virus | X | X | | | |
| | | Rubulavirus | Mumps virus | X | X | | | |
| | | Avulavirus | Human parainfluenxa | X | | | | |
| | | | virus 4A | | | | | |
| | | | Nipah virus | X | | | | |
| | | | Turkey rhinotracheitis | X | | | | |
| | | | virus | | | | | |
| | [Pneumovirinae] | Metapneumovirus | Pneumonia virus of | X | X | | | |
| | | | mice | | | | | |
| | Rhabdoviridae | Vesiculovirus | Vesicular stomatitis | X | | | | |
| | | | Indiana virus | | | | | |
| | | | Chandipura virus | X | X | | | |
| | | Lyssavirus | Rabies virus | X | X | | | |
| | | | Viral hemorrhagic | | X | | | |
| | | | septicemia virus | | | | | |
| | | | Sigma virus | | X | | | |
| | | Ephemerovirus | Bovine ephemeral fever | X | | | | |
| | | | virus | | | | | |
| | | Novirhabdovirus | Mokola virus | X | | | | |
| | | | Piryvirus | X | | | | |
| Mononegavirales | Filoviridae | “Marburg-like | Marburg virus | X | | | | |
| | | viruses” | | | | | | |
| | | “Ebola-like | Zaire Ebola virus | X | X | X | | |
| | | viruses” | | | | | | |
| | Orthomyxoviridae | Influenzavirus A | Influenza A virus | X | | | | |
| | | Influenzavirus C | Influenza C virus | X | | | | |
| | Bunyaviridae | Orthobunyavirus | California encephalitis | X | | | | |
| | | | virus | | | | | |
| | | | Mermet virus | X | | | | |
| | | Hantavirus | Puumala virus | | X | | | |
| | | | Khabarovsk virus | | X | | | |
| | | | Vladivostok virus | | X | | | |
| | | Nairovirus | Crimean Congo | | X | | | |
| | | | hemorragic fever virus | | | | | |
| | | Phlebovirus | Rift Valley fever virus | X | | | | |
| | | | Toscana virus | X | | | | |
| | Arenaviridae | Arenavirus | Lymphocytic | X | | | | |
| | | | choriomeningitis virus | | | | | |
| | | | Lassa virus | X | X | X | | |
| Group VI: RNA Reverse Transcribing Viruses | | | | | | | | |
| | Retroviridae | Alpharetrovirus | Avian leucosis virus | X | X | X | | |
| | | | Avian retrovirus RAV-O | X | | | | |
| | | | Avian myelocytomatosis | X | | | | |
| | | | virus | | | | | |
| | | | Avian sarcoma virus | X | | | | |
| | | | Avian erythroblastosis | X | | | | |
| | | | virus | | | | | |
| | | | Avian spleen necrosis | X | | | | |
| | | | virus | | | | | |
| | | | Fujinami sarcoma virus | X | | | | |
| | | | Rous sarcoma virus | X | | | | |
| | | Betaretrovirus | Mouse mammary tumour | X | | | | |
| | | | virus | | | | | |
| | | | Enzootic nasal tumor | X | X | X | | |
| | | | virus | | | | | |
| | | | Ovine enzootic nasal | X | X | X | | |
| | | | tumor virus | | | | | |
| | | | Sheep pulmonary | X | X | X | | |
| | | | adenomatosis virus | | | | | |
| | | | Simian Mason-Pfizer | X | | | | |
| | | | virus | | | | | |
| | | Gammaretrovirus | Murine leukeamia virus | X | X | X | | |
| | | | Gibbon Ape Leukemia | X | | | X | |
| | | | virus | | | | | |
| | | | Feline sarcoma virus | X | | | | |
| | | | Feline leukemia virus | X | | | | |
| | | | Murine sarcoma virus | X | | | | |
| | | | Simian sarcoma virus | X | | | | |
| | | Deltaretrovirus | Bovine leukaemia virus | X | | | | |
| | | | HTLV-I | X | X | X | | |
| | | | HTLV-II | X | X | | | |
| | | | STLV-I | X | X | X | | |
| | | Lentivirus | Human immunodeficiency | X | X | X | | X |
| | | | virus 1 | | | | | |
| | | | HIV-2 | X | X | | X | X |
| | | | CIV | X | X | X | X | X |
| | | | SIV | X | X | | X | X |
| | | | HTLV-4 | X | | | | |
| | | | BIV | X | | | | |
| | | Spumavirus | Chimpanzee foamy virus | X | X | | | |
| | | | Human spumavirus | X | X | | | |
| Group VII: DNA Reverse Transcribing Viruses | | | | | | | | |
| | Hepadnaviridae | Orthohepadnavirus | Hepatitis B virus | X | X | | | |
| | | Avihepadnavirus | Duck hepatitis B virus | X | | | | |
| | Caulimoviridae | Badnavirus | Commelina yellow mottle | X | | | | |
| | | | virus | | | | | |
| | | “Soybean chlorotic | Soybean chlorotic | X | | | | |
| | | mottle-like viruses” | mottle virus | | | | | |
| | | “Cassava vein | Cassava vein mosaic | X | | | | |
| | | mosaic-like viruses” | virus | | | | | |
| | | “Petunia vein | “Petunia vein | X | | | | |
| | | clearing-like | clearing virus | | | | | |
| | | viruses” | | | | | | |
| | | | Banana Streak virus | X | | | | |

The deletion mutation is a deletion of one or more amino acids from the budding mediating motif, such PTAP (SEQ ID NO:1) motif and/or PPXY (SEQ ID NO:2) motif and/or YXXL (SEQ ID NO:3) motif in viral proteins having late (L) domains which mediate the budding process, and optionally one or more amino acids surrounding the budding mediating motif in these proteins. The deletion mutations are prepared using standard techniques well known to persons skilled in the art. For example, a deletion mutation is prepared by restriction endonuclease digestion of the Gag encoding nucleic acid or the matrix protein encoding nucleic acid. Alternatively, deletion mutations are prepared using site-directed mutagenesis. One suitable site-directed mutagenesis technique is the QuikChange® site-directed mutagenesis protocol (Stratagene). This mutation can also be accomplished using standard polymerase chain reaction (PCR). Suitable protocols can be found in Current Protocols in Molecular Biology and Molecular Cloning textbooks, such as referenced herein. The preparation of these deletion mutations may be made using nucleic acid encoding the Gag protein or matrix protein or using vectors which contain the Gag protein or matrix protein encoding nucleic acids. Although any HIV-1 Gag p6 and SIV Gag nucleic acid can be used for these embodiments, it is preferred to use codon optimized nucleic acids. A codon optimized nucleic acid can be prepared as described in U.S. published patent application No. 20030223964, incorporated herein by reference. Briefly, the codon usage pattern of the nucleic acid, e.g., HIV-1 Gag, is modified so that the resulting nucleic acid sequence is comparable to the codon usage found in highly expressed human (or simian for SIV) genes. The modified nucleic acids provide improved expression of the encoded protein. An example of a codon optimized nucleic acid is HIV-1 Gag (Czm) (SEQ ID NO:10).

In a second aspect, the present invention provides a vector comprising a nucleic acid which comprises a deletion mutant of the budding mediating motif described herein. In one embodiment of this second aspect, the vector comprises a nucleic acid comprises a deletion mutant of the human immunodeficiency virus 1 (HIV-1) Gag p6 “PTAP” (SEQ ID NO:1) motif in HIV-1 Gag. In a second embodiment of this aspect, the vector comprises a nucleic acid comprises a deletion mutant of the simian immunodeficiency virus (SIV) Gag “PTAP” (SEQ ID NO:1) motif in SIV Gag. The vector may be any suitable vector known to persons skilled in the art for preparing or utilizing the deletion mutant of the budding mediating motif. For example, the vector may be a cloning vector which contains the HIV-1 Gag p6 nucleic acid or the SIV Gag encoding nucleic acid and which is used to prepare the deletion mutants of the present invention. The vector may also be a cloning vector which contains the mutant HIV-1 Gag p6 encoding nucleic acid or mutant SIV Gag encoding nucleic acid. Alternatively, the vector may be an expression vector which contains a nucleic acid encoding a deletion mutant of the present invention and which is used for expressing the deletion mutant of the present invention. For expression vectors, mammalian expression vectors are preferred.

The expression vectors of the invention comprise a nucleic acid of the invention in a form suitable for expression of the nucleic acid in a host cell, which means that the expression vectors include one or more regulatory sequences, selected on the basis of the host cells to be used for expression, which is operatively linked to the nucleic acid sequence to be expressed. The nucleotide sequence of interest is linked to the regulatory sequence(s) in a manner which allows for expression of the nucleotide sequence (e.g., in an in vitro transcription/translation system or in a host cell when the vector is introduced into the host cell). Regulatory sequences include promoters, enhancers and other expression control elements (e.g., polyadenylation signals). Such regulatory sequences are described, for example, in Goeddel (76). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector may depend on such factors as the choice of the host cell to be transformed, the level of expression of protein desired, etc. The expression vectors of the invention can be introduced into host cells to thereby produce proteins or peptides, including fusion polypeptides or peptides, encoded by nucleic acids as described herein.

In another embodiment, the mammalian expression vector is capable of directing expression of the nucleic acid preferentially in a particular cell type (e.g., tissue-specific regulatory elements are used to express the nucleic acid). Tissue-specific regulatory elements are known in the art. See, for example, U.S. Pat. No. 6,632,436, incorporated herein by reference. Non-limiting examples of suitable tissue-specific promoters include lymphoid-specific promoters (77), in particular promoters of T cell receptors (78) and immunoglobulins (79, 80).

In a third aspect, the present invention provides a vector, e.g., a plasmid, comprising a nucleic acid which comprises a deletion mutant of the budding mediating motif described herein and one or more nucleic acids, each of which encodes an additional structural or regulatory viral protein, including but not limited to, Pol, Env, Rev, Tat or Nef. Thus, the vector comprises a nucleic acid comprising the deletion mutant of the budding mediating motif and (a) a nucleic acid encoding Pol, Env, Rev, Tat or Nef, or (b) nucleic acids encoding two of Pol, Env, Rev, Tat and Nef, or (c) nucleic acids encoding three of Pol, Env, Rev, Tat and Nef, or (d) nucleic acids encoding four of Pol, Env, Rev, Tat and Nef or (e) nucleic acids encoding all of the following proteins: Pol, Env, Rev, Tat and Nef. An example of a nucleic acid sequence encoding HIV-1 Pol is Accession No. K03455. An example of a nucleic acid sequence encoding HIV-1 Env is Accession No. K03455. An example of a nucleic acid sequence encoding HIV-1 Rev is Accession No. AY496680 (locus AY496679S2). An example of a nucleic acid sequence encoding HIV-1 Tat is Accession No. AY496680 (locus AY496679S2). An example of a nucleic acid sequence encoding HIV-1 Nef is Accession No. AY496680 (locus AY496679S2). It is understood that the nucleic acid sequence of any HIV-1 isolate can be used in place of these specific sequences.

In one embodiment of this third aspect directed to HIV-1, the vector comprises a nucleic acid comprising a deletion mutant of the human immunodeficiency virus 1 (HIV-1) Gag p6 “PTAP” (SEQ ID NO:1) motif and a nucleic acid encoding one of HIV-1 Pol, HIV-1 Env, HIV-1 Rev, HIV-1 Tat or HIV-1 Nef. In a second embodiment of this third aspect directed to HIV-1, the vector comprises a nucleic acid comprising a deletion mutant of the human immunodeficiency virus 1 (HIV-1) Gag p6 “PTAP” (SEQ ID NO:1) motif and two nucleic acids encoding two of HIV-1 Pol, HIV-1 Env, HIV-1 Rev, HIV-1 Tat or HIV-1 Nef. In a third embodiment of this third aspect directed to HIV-1, the vector comprises a nucleic acid comprising a deletion mutant of the human immunodeficiency virus 1 (HIV-1) Gag p6 “PTAP” (SEQ ID NO:1) motif and three nucleic acids encoding three of HIV-1 Pol, HIV-1 Env, HIV-1 Rev, HIV-1 Tat or HIV-1 Nef. In a fourth embodiment of this third aspect directed to HIV-1, the vector comprises a nucleic acid comprising a deletion mutant of the human immunodeficiency virus 1 (HIV-1) Gag p6 “PTAP” (SEQ ID NO:1) motif and four nucleic acids encoding four of HIV-1 Pol, HIV-1 Env, HIV-1 Rev, HIV-1 Tat or HIV-1 Nef. In a fifth embodiment of this third aspect directed to HIV-1, the vector comprises a nucleic acid comprising a deletion mutant of the human immunodeficiency virus 1 (HIV-1) Gag p6 “PTAP” (SEQ ID NO:1) motif and five nucleic acids encoding all of the following HIV-I proteins: Pol, HIV-1 Env, HIV-1 Rev, HIV-1 Tat or HIV-1 Nef.

In a first embodiment of this third aspect directed to SIV, the vector comprises a nucleic acid comprising a deletion mutant of the simian immunodeficiency virus (SIV) Gag “PTAP” (SEQ ID NO:1) motif in SIV Gag and a nucleic acid encoding one of SIV Pol, SIV Env, SIV Rev, SIV Tat or SIV Nef. In a second embodiment of this third aspect directed to SIV, the vector comprises a nucleic acid comprising a deletion mutant of the simian immunodeficiency virus (SIV) Gag p6 “PTAP” (SEQ ID NO:1) motif and two nucleic acids encoding two of SIV Pol, SIV Env, SIV Rev, SIV Tat or SIV Nef. In a third embodiment of this third aspect directed to SIV, the vector comprises a nucleic acid comprising a deletion mutant of the simian immunodeficiency virus (SIV) Gag p6 “PTAP” (SEQ ID NO:1) motif and three nucleic acids encoding three of SIV Pol, SIV Env, SIV Rev, SIV Tat or SIV Nef. In a fourth embodiment of this third aspect directed to SIV, the vector comprises a nucleic acid comprising a deletion mutant of the simian immunodeficiency virus (SIV) Gag p6 “PTAP” (SEQ ID NO:1) motif and four nucleic acids encoding four of SIV Pol, SIV Env, SIV Rev, SIV Tat or SIV Nef. In a fifth embodiment of this third aspect directed to SIV, the vector comprises a nucleic acid comprising a deletion mutant of the simian immunodeficiency virus (SIV) Gag p6 “PTAP” (SEQ ID NO:1) motif and five nucleic acids encoding all of the following SIV proteins: Pol, SIV Env, SIV Rev, SIV Tat or SIV Nef.

The vector may be any suitable vector known to persons skilled in the art for preparing or utilizing the deletion mutant of the budding mediating motif. For example, the vector may be a cloning vector which contains the HIV-1 Gag p6 nucleic acid or the SIV Gag encoding nucleic acid and which is used to prepare the deletion mutants of the present invention. The vector may also be a cloning vector which contains the mutant HIV-1 Gag p6 encoding nucleic acid or mutant SIV Gag encoding nucleic acid. Alternatively, the vector may be an expression vector which contains a nucleic acid encoding a deletion mutant of the present invention and which is used for expressing the deletion mutant of the present invention. For expression vectors, mammalian expression vectors are preferred.

The expression vectors of the invention comprise a nucleic acid of the invention in a form suitable for expression of the nucleic acid in a host cell, which means that the expression vectors include one or more regulatory sequences, selected on the basis of the host cells to be used for expression, which is operatively linked to the nucleic acid sequence to be expressed. The nucleotide sequence of interest is linked to the regulatory sequence(s) in a manner which allows for expression of the nucleotide sequence (e.g., in an in vitro transcription/translation system or in a host cell when the vector is introduced into the host cell). Regulatory sequences include promoters, enhancers and other expression control elements (e.g., polyadenylation signals). Each nucleic acid sequence may be under control of a single promoter or may be under control of separate promoters, which may be the same. Such regulatory sequences are described, for example, in Goeddel (76). Regulatory sequences include those which direct constitutive expression of a nucleotide sequence in many types of host cell, such as the cytomegalovirus (CMV) promoter, and those which direct expression of the nucleotide sequence only in certain host cells (e.g., tissue-specific regulatory sequences), such as those described below. It will be appreciated by those skilled in the art that the design of the expression vector may depend on such factors as the choice of the host cell to be transformed, the level of expression of protein desired, etc. The expression vectors of the invention can be introduced into host cells to thereby produce proteins or peptides, including fusion polypeptides or peptides, encoded by nucleic acids as described herein.

In another embodiment, the mammalian expression vector is capable of directing expression of the nucleic acid preferentially in a particular cell type (e.g., tissue-specific regulatory elements are used to express the nucleic acid). Tissue-specific regulatory elements are known in the art. See, for example, U.S. Pat. No. 6,632,436, incorporated herein by reference. Non-limiting examples of suitable tissue-specific promoters include lymphoid-specific promoters (77), in particular promoters of T cell receptors (78) and immunoglobulins (79, 80).

In a fourth aspect, the present invention provides a nucleic acid comprising a molecular clone of a virus with a deletion the budding mediating motif and to a vector containing such a nucleic acid. The mutant budding mediating motif sequences are prepared as described above. Suitable vectors include those previously described, but which contain the molecular clone of the virus with a deletion mutant in the budding mediating motif. In one embodiment of this fourth aspect, the nucleic acid comprises a molecular clone of HIV-1, such as the HXB2 molecular clone (Accession No. K03455), with a deletion in the Gag “PTAP” (SEQ ID NO:1) motif and to a vector, e.g., a plasmid, containing such a nucleic acid. In a second embodiment of this fourth aspect, the nucleic acid comprises a molecular clone of SIV, such as the SIV251 molecular clone (Accession No. M19499), with a deletion in the Gag “PTAP” (SEQ ID NO:1) motif and to a vector, e.g., a plasmid, containing such a nucleic acid.

In a fifth aspect, the present invention provides products produced by these nucleic acids, e.g., mRNA, protein and viral particles. Using a CMV promoter or other strong, high efficiency, promoter instead of the native viral promoter (such as HIV-1 LTR promoter for the HIV constructs) in a packaging vector (such as those well known to persons skilled in the art), high transcription of the mutant nucleic acids described herein and high expression of the mutant viral proteins with the mutant budding mediating motif described herein can be achieved in the total absence of any other viral protein. The exchange of the native viral promoter with other promoters is beneficial in the packaging vector or other vectors if constitutive expression is desirable and also for expression in other mammalian cells, such as mouse cells, in which the native viral promoter is weak. Vectors containing the mutant viral protein encoding sequences described herein such as the mutant Gag sequences of the present invention can be used for the independent production of the mutant viral proteins described herein, such as the HIV-1 Gag p6 and mutant SIV Gag proteins. In addition, vectors containing the mutant viral protein encoding sequences described herein, such as the mutant Gag sequences of the invention, and the additional sequences, e.g., pol, env, rev, tat, nef, etc., can be used for the independent production of the mutant viral proteins described herein, such as the mutant HIV-1 Gag p6 and mutant SIV Gag proteins, and the additionally encoded proteins, e.g., pol, env, etc.

The group-specific antigens (Gag) of human immunodeficiency virus type-1 (HIV-1) self-assemble into noninfectious virus-like particles (VLP) that are released from various eucaryotic cells by budding (81). The synthetic expression cassettes of the present invention provide efficient means for the production of HIV-mutant Gag p6 or SIV-mutant Gag virus-like particles (VLPs) using a variety of different cell types, including, but not limited to, mammalian cells.

VLPs can spontaneously form when the particle-forming polypeptide of interest is recombinantly expressed in an appropriate host cell. Thus, the VLPs produced using the expression vectors of the present invention are conveniently prepared using recombinant techniques. As discussed above the mutant Gag polypeptide encoding expression vectors of the present invention can include other polypeptide coding sequences of interest (e.g., pol, env, etc.). Expression of such expression vectors yields VLPs comprising the product of the synthetic expression cassette, as well as, the polypeptide of interest.

Depending on the expression system and host selected, the VLPs are produced by growing host cells transformed by an expression vector under conditions whereby the particle-forming polypeptide is expressed and VLPs can be formed. The selection of the appropriate growth conditions is within the skill of the art. If the VLPs are formed intracellularly, the cells are then disrupted, using chemical, physical or mechanical means, which lyse the cells yet keep the VLPs substantially intact. Such methods are known to those of skill in the art and are described in, e.g., Harris and Angal (82). The particles are then isolated (or substantially purified) using methods that preserve the integrity thereof, such as, by density gradient centrifugation, e.g., sucrose gradients, PEG-precipitation, pelleting, and the like (see, e.g., Kimbauer et al. (83)), as well as standard purification techniques including, e.g., ion exchange and gel filtration chromatography. See also, U.S. published patent application No. 20030223964, incorporated herein by reference.

The proteins and VLPs produced in accordance with the present invention can be used to detect antibodies to HIV-1 or SIV using techniques well known to persons skilled in the art.

In a sixth aspect, the present invention provides compositions comprising these nucleic acids and/or their expression products. The nucleic acids, expression vectors, expression products and VLPs of the present invention are formulated into compositions for delivery to the vertebrate subject. These compositions may either be prophylactic (to prevent infection) or therapeutic (to treat disease after infection). The compositions will comprise a “therapeutically effective amount” of the gene of interest such that an amount of the antigen can be produced in vivo so that an immune response is generated in the individual to which it is administered. The exact amount necessary will vary depending on the subject being treated; the age and general condition of the subject to be treated; the capacity of the subject's immune system to synthesize antibodies; the degree of protection desired; the severity of the condition being treated; the particular antigen selected and its mode of administration, among other factors. An appropriate effective amount can be readily determined by one of skill in the art. Thus, a “therapeutically effective amount” will fall in a relatively broad range that can be determined through routine trials.

The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Certain facilitators of immunogenicity or of nucleic acid uptake and/or expression can also be included in the compositions or coadministered, such as, but not limited to, bupivacaine, cardiotoxin and sucrose.

In a seventh aspect, the present invention provides host cells (e.g., packaging cells) comprising these nucleic acids, their expression products, vectors or viral-like particles. For example, in one embodiment, packaging cell line are provided comprising an expression vector of the present invention. Packaging cell lines may further comprise a promoter and a sequence encoding Tat, Rev, or an Env, wherein the promoter is operably linked to the sequence encoding Tat, Rev, or the Env. The packaging cell line may further comprise a sequence encoding any one or more of Nef, Vif, Vpu or Vpr.

Briefly, the parent cell line from which the packaging cell line is derived can be selected from a variety of mammalian cell lines, including for example, 293, RD, COS-7, CHO, BHK, VERO, HT1080, and myeloma cells. After selection of a suitable host cell for the generation of a packaging cell line, one or more expression vectors are introduced into the cell line in order to complement or supply in trans components of the virus which have been deleted. Further, such packaging cell lines can also be used to produce VLPs.

In addition to packaging cells, a second embodiment is any host cell which can be used to produce the protein encoded by the nucleic acids of the present invention. Such cells may be mammalian cells, such as those identified above. Other cells, cell types, tissue types, etc., that may be useful in the practice of the present invention include, but are not limited to, those obtained from the following: insects (e.g., Trichoplusia ni (Tn5) and Sf9), bacteria, yeast, plants, antigen presenting cells (e.g., macrophage, monocytes, dendritic cells, B-cells, T-cells, stem cells, and progenitor cells thereof), primary cells, immortalized cells, tumor-derived cells. These cells are transfected with the nucleic acids or expression vectors of the present invention using techniques well know to persons skilled in the art.

In an eighth aspect, the present invention provides a vaccine comprising the above described nucleic acids or vectors. The vaccine comprises a nucleic acid or an expression vector of the present invention in which the nucleic acid encodes a mutant HIV-1 Gag p6 or mutant SIV Gag described herein. As described herein, the nucleic acid is amenable to translation by the eukaryotic cellular machinery (ribosomes, tRNAs, and other translation factors). Where the protein encoded by the polynucleotide is one which does not normally occur in that animal except in pathological conditions, (i.e., a heterologous protein) such as proteins associated with HIV or SIV, the etiologic agent of acquired immune deficiency syndrome, (AIDS), the animals” immune system is activated to launch a protective immune response. Because these exogenous proteins are produced by the animals' tissues, the expressed proteins are processed by the major histocompatibility system, MHC, in a fashion analogous to when an actual infection with the related organism occurs. The result, as shown in this disclosure, is induction of immune responses against the cognate pathogen.

Accordingly, the nucleic acids of the present invention which, when introduced into the biological system induce the expression of the mutant Gag protein, as well as any additional proteins which may be encoded by the nucleic acid, and epitopes. The induced antibody response is both specific for the expressed proteins, and neutralizes HIV. In addition, cytotoxic T-lymphocytes which specifically recognize and destroyHIV infected cells are induced. The dual humoral and cellular immune responses generated according to this invention are particularly significant to inhibiting HIV infection, given the propensity of HIV to mutate within the population, as well as in infected individuals.

The nucleic acid in the vaccine may be naked, that is, unassociated with any proteins, adjuvants or other agents which impact on the recipients' immune system. In this case, it is desirable for the polynucleotide to be in a physiologically acceptable solution, such as, but not limited to, sterile saline or sterile buffered saline. Alternatively, the DNA may be associated with liposomes, such as lecithin liposomes or other liposomes known in the art, as a DNA-liposome mixture, or the DNA may be associated with an adjuvant known in the art to boost immune responses, such as a protein or other carrier. Agents which assist in the cellular uptake of DNA, such as, but not limited to, calcium ions, may also be used to advantage. These agents are generally referred to herein as transfection facilitating reagents and pharmaceutically acceptable carriers. Techniques for coating microprojectiles coated with polynucleotide are known in the art and are also useful in connection with this invention.

The HIV-peptides or HIV-encoded DNA constructs of the present invention may be formulated into the vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The amount of expressible DNA to be introduced into a vaccine recipient will depend on the strength of the transcriptional and translational promoters used and on the immunogenicity of the expressed gene product. In general, an immunologically or prophylactically effective dose of about 1 ng to 100 mg, and preferably about 10 μg to 300 μg is administered directly into muscle tissue. Subcutaneous injection, intradermal introduction, impression through the skin, and other modes of administration such as intraperitoneal, intravenous, or inhalation delivery are also contemplated. It is also contemplated that booster vaccinations are to be provided. Following vaccination with HIV polynucleotide immunogen, boosting, as described further below is also contemplated. Parenteral administration, such as intravenous, intramuscular, subcutaneous or other means of administration of interleukin-12 protein or GM-CSF or similar proteins alone or in combination, concurrently with or subsequent to parenteral introduction of the vaccine of this invention is also advantageous.

In a ninth aspect, the present invention provides a method for immunization by administering an effective amount of the vaccine provided by the invention. Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The vaccines are administered in a manner compatible with the dosage formulation, and in such amount as will be therapeutically effective and immunogenic. The quantity to be administered depends on the subject to be treated, including, e.g., the capacity of the individual's immune system to synthesize antibodies and the degree of protection desired. Precise amounts of active ingredient required to be administered depend on the judgment of the practitioner. However, suitable dosage ranges are as described herein. Suitable regimes for initial administration and booster shots are also variable, but are typified by an initial administration followed by subsequent inoculations or other administrations.

The vaccines of this invention may be administered alone, or may be part of a prime and boost administration regimen. A mixed modality priming and booster inoculation scheme will result in an enhanced immune response. This one aspect of this invention is a method of priming a subject with the DNA vaccine by administering the vaccine at least one time, allowing a predetermined length of time to pass, and then boosting by administering the boost. Multiple primings, typically, 1-4, are usually employed, although more may be used. The length of time between priming and boost may typically vary from about four months to a year, but other time frames may be used.

In a tenth aspect, the present invention provides a method for augmenting a cellular immune response to an immunodeficiency virus by administering an effective amount of the vaccine provided by the present invention. As demonstrated herein, the administration of a DNA vaccine of the present invention, optionally with boosting, increases the cell mediated immune (CMI) response to HIV or SIV in the treated subject. The augmented cellular immune response is useful for the be prophylaxis (prevention of infection) or therapy (treatment of disease after infection), e.g. as an aid for antiviral therapy.

CMI responses elicited by vaccines in mice and nonhuman primates are monitored using several assays, including cytokine production, lymphocyte proliferation and cytotoxic lymphocyte activity in vaccinated animals.

Cytokines are pleiotropic molecules that play critical roles in activation, growth, apoptosis, differentiation, inflammation, and in host anti-viral defense; hence, much work has been dedicated to understanding their regulation (28-31). Cytokines serve as key markers in identifying whether naïve CD4+ and CD8+ T cells have differentiated to Th1/ Th2 or Tc1/Tc2 lineages, respectively. This information is of importance given that a coordinated effort of both humoral and cellular immune responses, resulting from the activities of both CD4+ and CD8+ T cells, is necessary in combatting HIV-1. Helper T cell function elicited by differentiated CD4+ Th1 (IFNγ and TNFα-producing) cells serve to augment the CD8+ CTL activity and hence, bolster CMI responses. Conversely, differentiated CD4+ Th2 (IL4, IL5, IL10-producing) cells contribute to antibody production and an increased humoral response. Moreover, the characteristic production of IFNγ and TNFα in CD8+ T Icells has been shown to correlate with cytolytic activity and thus can be used to evaluate the CMI response elicited by candidate vaccines.

An intracellular cytokine staining (ICC) protocol has been developed and optimized to assess the production of Th1 (IFNγ and TNFα) and Th2 (IL4, IL5, IL10) cytokines in both mice and nonhuman primates. The immunogenicity of candidate vaccines can therefore be tested based on the spectrum of cytokines that are produced in both CD4+ and CD8+ T cells following antigen re-stimulation ex-vivo and analysis of cells by flow cytometry. An IFNγ enzyme-linked immunospot assay has been developed and optimized for the detection of secreted IFNγ from lymphocytes. This method offers a powerful tool for detecting and enumerating individual immune cells that secrete IFNγ in vitro. Details of these methods are provided in the Examples.

Lymphocyte proliferation assays represent useful and widely employed tools in evaluating the functional capabilities of lymphocytes. These assays provide a simple semi-quantitative in vitro correlate with cell mediated immunity. Lymphocyte proliferation occurs when cells are cultured with the specific antigen to which the animal was previously exposed or vaccinated. Thus, naïve animals not previously exposed to a particular antigen, will not respond to the antigen. The method that has been developed to evaluate lymphocyte proliferation uses the cell-permeant dye, carboxyfluorescein diacetate succinimidyl ester (CFSE) that covalently attaches to cytoplasmic components of cells, resulting in uniform fluorescence. Upon cell division, the dye is distributed equally between daughter cells, allowing the resolution of up to eight cycles of cell division by flow cytometry. Details of this method are provided in the Examples.

CTL are well known for their role in the initial clearance of primary viremia in HIV-1 infection. Hence, in evaluating the immunogenicity of candidate HIV-1 vaccines, a direct measure of the CD8+ CTL activity is a necessary parameter. A flow cytometry-based CTL (FCC) assay first described by Liu et al (32) has been developed to monitor and quantify antigen specific target cell killing activities mediated by CTL. This non-radioactive assay relies on CTL-induced caspase activation in target cells. Detection of the specific cleavage of fluorogenic caspase substrates is accomplished using flow cytometry. Varying effector-to-target cell ratios are tested using antigen re-stimulated effector cells from splenocytes of vaccinated animals and peptide-loaded P815 target cells. This assay enables antigen-specific cellular immune responses to be measured in real-time at the single cell level. Details of this method are provided in the Examples.

Classic references for DNA vaccines include the first demonstration of the raising of an immune response (33); the first demonstration of cytotoxic T-cell (Tc)-mediated immunity (34); the first demonstration of the protective efficacy of intradermal (i.d.), intramuscular (i.m.), intravenous (i.v.), intranasal (i.n.), and gene gun (g.g.) immunizations (35, 36); the first use of genetic adjuvants (37); the first use of library immunizations (38); and the first demonstration of the ability to modulate the T-helper type of an immune response by the method of DNA delivery (39).

The efficacy of a vaccine is measured by the extent of protection against a later challenge by a pathogen. Effective vaccines are immunogens that can induce high titer and long-lasting protective immunity for targeted intervention against diseases after a minimum number of inoculations. For example, genetic immunization is an approach to elicit immune responses against specific proteins by expressing genes encoding the proteins in an animal's own cells. The substantial antigen amplification and immune stimulation resulting from prolonged antigen presentation in vivo can induce a solid immunity against the antigen. Genetic immunization simplifies the vaccination protocol to produce immune responses against particular proteins because the often difficult steps of protein purification and combination with adjuvant, both routinely required for vaccine development, are eliminated. Since genetic immunization does not require the isolation of proteins, it is especially valuable for proteins that may lose conformational epitopes when purified biochemically. Genetic vaccines may also be delivered in combination without eliciting interference or affecting efficacy (67, 68), which may simplify the vaccination scheme against multiple antigens.

The major immunological advantage of DNA-based immunizations is the ability of the immunogen to be presented by both MHC class I and class II molecules. Endogenously synthesized proteins readily enter processing pathways for the loading of peptide epitopes onto MHC I as well as MHC II molecules. MHC I-presented epitopes raise cytotoxic T-cells (Tc) responses whereas MHC II-presented epitopes raise helper T-cells (Th). By contrast, immunogens that are not synthesized in cells are largely restricted to the loading of MHC II epitopes and the raising of Th but not Tc. When compared with live attenuated vaccines or recombinant viral vectors that produce immunogens in cells and raise both Th and Tc, DNA vaccines have the advantages of not being infectious and of focusing the immune response on only those antigens desired for immunization. DNA vaccines also are advantageous because they can be manipulated relatively easily to raise type 1 or type 2 T-cell help. This allows a vaccine to be tailored for the type of immune response that will be mobilized to combat an infection. DNA vaccines are also cost effective because of the ease with which plasmids can be constructed using recombinant DNA technology, the ability to use a generic method for vaccine production (growth and purification of plasmid DNA), and the stability of DNA over a wide range of temperatures.

The best immune responses are achieved using highly active expression vectors modeled on those developed for the production of recombinant proteins (40). The most frequently used transcriptional control elements include a strong promoter. One such promoter suitable for use is the cytomegalovirus (CMV) intermediate early promoter, although other promoters may be used in a DNA vaccine without departing from the scope the present invention. Other transcriptional control elements useful in the present invention include a strong polyadenylation signal such as, for example, that derived from a bovine growth hormone encoding gene, or a rabbit beta. globin polyadenylation signal (41-45). The CMV immediate early promoter may be used with or without intron A (42). The presence of intron A increases the expression of many antigens from RNA viruses, bacteria, and parasites, presumably by providing the expressed RNA with sequences which support processing and function as an eukaryotic mRNA. It will be appreciated that expression also may be enhanced by other methods known in the art including, but not limited to, optimizing the codon usage of prokaryotic mRNAs for eukaryotic cells (46, 47). Multi-cistronic vectors may be used to express more than one immunogen or an immunogen and a immunostimulatory protein (48, 49).

Two approaches to DNA delivery are injection of DNA in saline using a hypodermic needle or gene gun delivery of DNA-coated gold beads. Saline injections deliver DNA into extracellular spaces, whereas gene gun deliveries bombard DNA directly into cells. The saline injections require much larger amounts of DNA (100-1000 times more) than the gene gun (35). These two types of delivery also differ in that saline injections bias responses towards type 1 T-cell help, whereas gene gun deliveries bias responses towards type 2 T-cell help (39, 50). DNAs injected in saline rapidly spread throughout the body. DNAs delivered by the gun are more localized at the target site. Following either method of inoculation, extracellular plasmid DNA has a short half life on the order of 10 minutes (51, 52). Vaccination by saline injections can be intramuscular (i.m.) or intradermal (i.d.) (35).

Although intravenous and subcutaneous injections have met with different degrees of success for different plasmids (53, 35), intraperitoneal injections have not met with success (53, 35). Gene gun deliveries can be administered to the skin or to surgically exposed muscle. Methods and routes of DNA delivery that are effective at raising immune responses in mice are effective in other species.

Immunization by mucosal delivery of DNA has been less successful than immunizations using parenteral routes of inoculation. Intranasal administration of DNA in saline has met with both good (54, 55) and limited (35) success. The gene gun has successfully raised IgG following the delivery of DNA to the vaginal mucosa (56). Some success at delivering DNA to mucosal surfaces has also been achieved using liposomes (57), microspheres (58, 59) and recombinant Shigella vectors (60, 61).

Vectors carrying an expression cassette of the present invention are formulated into compositions for delivery to the vertebrate subject. These compositions may either be prophylactic (to prevent infection) or therapeutic (to treat disease after infection). The compositions will comprise a “therapeutically effective amount” of the gene of interest such that an amount of the antigen can be produced in vivo so that an immune response is generated in the individual to which it is administered. An appropriate effective amount can be readily determined by one of skill in the art. Thus, a “therapeutically effective amount” will fall in a relatively broad range that can be determined through routine trials.

Typically, the dose of DNA needed to raise a response depends upon the method of delivery, the host, the vector, and the encoded antigen. The most profound effect is seen for the method of delivery. From 10 μg to 1 mg of DNA is generally used for saline injections of DNA, whereas from 0.2 μg to 20 μg of DNA is used for gene gun deliveries of DNA. In general, lower doses of DNA are used in mice (10-100 μg for saline injections and 0.2 μg to 2 μg for gene gun deliveries), and higher doses in primates (100 μg to 1 mg for saline injections and 2 μg to 20 μg for gene gun deliveries). The much lower amount of DNA required for gene gun deliveries reflect the gold beads directly delivering DNA into cells.

An example of the marked effect of an antigen on the raised response can be found in studies comparing the ability to raise antibody responses in rabbits of DNAs expressing the influenza hemagglutinin or an immunodeficiency virus envelope glycoprotein (Env) (62). Under similar immunization conditions, the hemagglutinin-expressing DNA raised long lasting, high avidity, high titer antibody (about 100 μg per ml of specific antibody), whereas the Env-expressing DNA raised only transient, low avidity, and low titer antibody responses (<10 μg per ml of specific antibody). These differences in raised antibody were hypothesized to reflect the hemagglutinin being a T-dependent antigen and the highly glycosylated immunodeficiency virus Env behaving as a T-independent antigen.

Both protein and recombinant viruses have been used to boost DNA-primed immune responses. Protein boosts have been used to increase neutralizing antibody responses to the HIV-1 Env. Recombinant pox virus boosts have been used to increase both humoral and cellular immune responses.

For weak immunogens, such as the immunodeficiency virus Env, for which DNA-raised antibody responses are only a fraction of those in naturally infected animals, protein boosts have provided a means of increasing low titer antibody responses (62, 63). In a study in rabbits, the protein boost increased both the titers of antibody and the avidity and the persistence of the antibody response (62). Consistent with a secondary immune response to the protein boost, DNA primed animals showed both more rapid increases in antibody, and higher titers of antibody following a protein boost than animals receiving only the protein. However, by a second protein immunization, the kinetics and the titer of the antibody response were similar in animals that had, and had not, received DNA priming immunizations.

Recombinant pox virus boosts have also proved to be a highly successful method of boosting DNA-primed CD8+ cell responses (64-66). Following pox virus boosters, antigen-specific CD8+ cells have been increased by as much as 10-fold in DNA primed mice or macaques. Studies testing the order of immunizations reveal that the DNA must be delivered first (66). This has been hypothesized to reflect the DNA focusing the immune response on the desired immunogens. The larger increases in CD8+ cell responses following pox virus boosts has been hypothesized to reflect both the larger amount of antigen expressed by the pox virus vector, as well as pox virus-induced cytokines augmenting immune responses (65, 66).

The nucleic acids or vectors of the present invention can be administered to a patient in the presence of adjuvants or other substances that have the capability of promoting DNA uptake or recruiting immune system cells to the site of the inoculation. Embodiments include combining the DNA vaccine with conventional adjuvants or genetic adjuvants. Conventional adjuvants, including reagents that favor the stability and uptake of the DNA, recruit immune system cells to the site of inoculation, or facilitate the immune activation of responding lymphoid cells, include but are not limited to oil and water emulsions (for example, complete Freund's adjuvant and incomplete Freund's adjuvant), Corynebacterium parvum, Bacillus Calmette Guerin, aluminum hydroxide, glucan, dextran sulfate, iron oxide, sodium alginate, Bacto-Adjuvant, certain synthetic polymers such as poly amino acids and co-polymers of amino acids, saponin, “REGRESSIN” (Vetrepharm, Athens, Ga.), “AVRIDINE” (N,N-dioctadecyl-N′,N′-bis(2-hydroxyethyl)-propanediamine), paraffin oil, and muramyl dipeptide. The present invention also contemplates the use of genetic adjuvants such as immunomodulatory molecules encoded in a co-inoculated DNA. The co-inoculated DNA can be in the same vaccine construct as the vaccine immunogen or in a separate DNA vector.

A vaccine according to the present invention can be administered in a variety of ways including through any parenteral or topical route. For example, an individual can be inoculated by intravenous, intraperitoneal, intradermal, subcutaneous or intramuscular methods. Inoculation can be, for example, with a hypodermic needle, needleless delivery devices such as those that propel a stream of liquid into the target site, or with the use of a gene gun that bombards DNA on gold beads into the target site. The vector comprising the pathogen vaccine insert can be administered to a mucosal surface by a variety of methods including intranasal administration, i.e., nose drops or inhalants, or intrarectal or intravaginal administration by solutions, gels, foams, or suppositories. Alternatively, the vector comprising the vaccine insert can be orally administered in the form of a tablet, capsule, chewable tablet, syrup, emulsion, or the like. In an alternate embodiment, vectors can be administered transdermally, by passive skin patches, iontophoretic means, and the like.

Any appropriate physiologically acceptable medium is suitable for introducing the vector comprising the pathogen vaccine insert into the patient. For example, suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like. Certain facilitators of immunogenicity or of nucleic acid uptake and/or expression can also be included in the compositions or coadministered, such as, but not limited to, bupivacaine, cardiotoxin and sucrose.

Immune responses induced by the polynucleotide vaccine constructs of the present invention can be demonstrated in mice, rabbits and primates. Mice provide the most facile animal model suitable for testing CMI induction by the constructs and are therefore used to evaluate whether a particular construct is immunogenic. Monitoring antibody production in rabbits also allows confirmation that a given construct is suitably immunogenic i.e., a high proportion of vaccinated animals show an antibody response. Nonhuman primates provide a suitable model system for evaluation of both CMI and humoral (antibody) immune responses. These species are also preferred to mice for antisera neutralization assays due to high levels of endogenous neutralizing activities against retroviruses observed in mouse sera. Sufficient immunogenicity is engendered by the vaccines of the present invention to achieve protection in experiments in a primate challenge model based upon known protective levels of neutralizing antibodies for this system. However, the currently emerging and increasingly accepted definition of protection in the scientific community is moving away from so-called “sterilizing immunity”, which indicates complete protection from HIV infection, to prevention of disease. A number of correlates of this goal include reduced blood viral titer, as measured either by HIV reverse transcriptase activity, by infectivity of samples of serum, by ELISA assay of p24 or other HIV antigen concentration in blood, increased CD4+ T-cell concentration, and by extended survival rates (see, for example, Cohen, J. (69) for a discussion of the evolving definition of anti-HIV vaccine efficacy). The immunogens of the instant invention also generate neutralizing immune responses against infectious (clinical, primary field) isolates of HIV.

### EXAMPLES

The present invention is described by reference to the following Examples, which are offered by way of illustration and are not intended to limit the invention in any manner. The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pennsylvania: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., Blackwell Scientific Publications, 1986); Sambrook and Russell, Molecular Cloning: A Laboratory Manual, 3rd Ed. (Cold Spring Harbor Laboratory Press, 2001); Short Protocols in Molecular Biology, 4th Ed. (Ausubel et al. eds., John Wiley & Sons, 1999); Molecular Biology Techniques: An Intensive Laboratory Course, (Ream et al., eds., Academic Press, 1998); PCR (Introduction to Biotechniques Series), 2nd Ed. (Newton & Graham eds., Springer Verlag, 1997).

### Example 1

### Assays to Monitor CMI Responses

Cytokine Production (Intracellular Cytokine Staining (ICC)): Cytokines represent a superfamily of signaling proteins that mediate a variety of biological functions. Comprised of interleukins, tumor necrosis factors, interferons, colony stimulating factors, and chemokines, these pleiotropic molecules play critical roles in activation, growth, apoptosis, differentiation, inflammation, and in host anti-viral defense. Cytokines mediate their effects on different cell populations via their cognate receptors on varied cell types. High affinity binding to specific cell surface receptors enables cytokines to transduce intracellular signals that can result in immunostimulatory effects. The importance of cytokines in T lymphocyte differentiation cannot be overstated. In addition, cytokines serve as key markers in identifying whether naïve CD4 and CD8 T cells have differentiated to Th1/Th2 or Tc1/Tc2 lineages, respectively. This information is of importance given that a coordinated effort of both humoral and cellular immune responses, resulting from the activities of both CD4 and CD8 T cells, is necessary in combatting HIV. The helper T cell function elicited from differentiated CD4 Th1 (IFNγ and TNFα-producing T cells) will serve to augment the CD8 CTL activity and hence bolster the CMI response. Conversely, differentiated CD4 Th2 (IL4, IL5, IL10 producing) cells will contribute to antibody production and an augmented humoral response. Moreover, the characteristic production of IFNγ and TNFα (Th1, Tc1) cytokines in CD8 T lymphocytes has been shown to correlate with a greater cytolytic activity and thus can be used to evaluate the CMI response elicited by candidate vaccines. ABL has developed and optimized an intracellular cytokine staining (ICC) protocol to assess the production of Th1 (IFNγ and TNFα) and Th2 (IL4, IL5, IL10) cytokines in the nonhuman primate model systems. The immunogenicity of candidate vaccines can therefore be tested based on the spectrum of cytokines that are produced in both CD4 and CD8 T cells following antigen re-stimulation ex-vivo and analysis of cells by flow cytometry.

Lymphocyte Proliferation: Lymphocyte proliferation assays represent useful and widely employed tools in evaluating the functional capabilities of lymphocytes. These assays provide a simple semi-quantitative in vitro correlate with cell mediated immunity. Lymphocyte proliferation occurs when cells are cultured with the specific antigen to which the animal was previously exposed or vaccinated. Thus, naïve animals not previously exposed to a particular antigen, will not respond to the antigen. Lymphocyte proliferation assays are routinely used to assess and monitor disease progression in HIV-infected individuals and to evaluate the degree of immune reconstitution following anti-retroviral therapy. Hence, these proliferation assays (recall antigen responses) may be used in evaluating the antigenicity of candidate vaccines.

As previously asserted, efficient CMI responses represent a coordinated effort from both CD4 and CD8 T cell subsets. Therefore, in evaluating the immunogenicity of anti-HIV candidates, ABL will assess the clonal proliferation of CD4 and CD8 T lymphocytes after short-term culture with recall antigen, alloantigen, or mitogens (PHA, PMA+ lonomycin). This will reveal the functional capabilities and relative contributions of both CD4 and CD8 T lymphocytes. The method that ABL has developed to evaluate lymphocyte proliferation uses the cell-permeant dye, carboxyfluorescein diacetate succinimidyl ester (CFSE) that covalently attaches to cytoplasmic components of cells, resulting in uniform fluorescence. Upon cell division, the dye is distributed equally between daughter cells, allowing the resolution of up to eight cycles of cell division by flow cytometry.

Cytotoxic T Lymphocyte (CTL) Activity: CTL are well known for their role in the initial clearance of primary viremia in HIV-1 infection. Hence, in evaluating the immunogenicity of candidate HIV-1 vaccines, a direct measure of the CD8+ CTL activity is a necessary parameter. We have developed the flow cytometry-based CTL (FCC) assay first described by Liu et al (32) to monitor and quantify antigen specific target cell killing activities mediated by CTL. This non-radioactive assay relies on CTL-induced caspase activation in target cells. Detection of the specific cleavage of fluorogenic caspase substrates is accomplished using flow cytometry. Varying effector-to-target cell ratios are tested using antigen re-stimulated effector cells from splenocytes of vaccinated animals (Balb/c mice) and peptide-loaded P815 target cells. This methodology is currently being developed for evaluating CTL activity in nonhuman primates. This assay enables antigen-specific cellular immune responses to be measured in real-time at the single cell level.

### Example 2

### Preparation of an HIV-1 Gag P6 Mutant

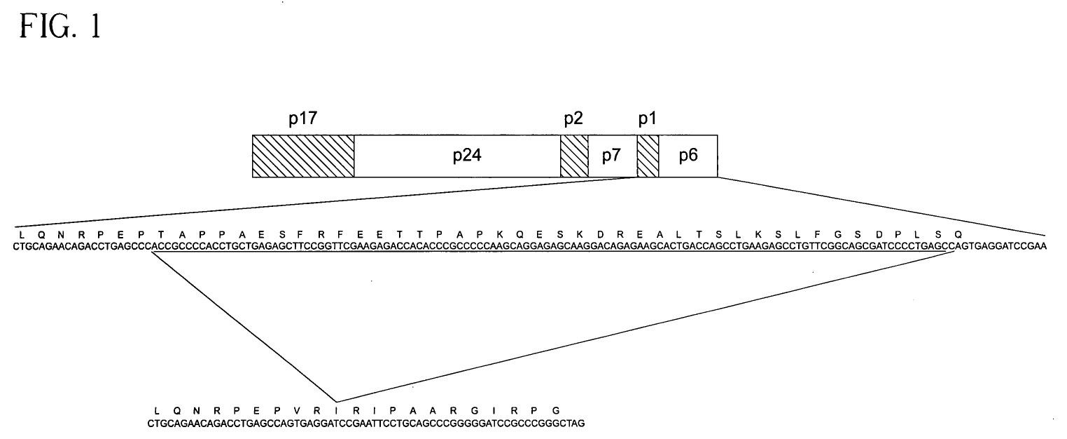

An HIV-1 Gag DNA vaccine was prepared in which the p6 region (containing the PTAP (SEQ ID NO:1) sequence motif) was deleted (FIG. 1). To this end, a codon optimized HIV-1 Gag (Czm) (SEQ ID NO:10), previously subcloned into a mammalian expression vector, was used for preliminary studies. This construct, GagA7, was found to contain two Bpu10I restriction enzyme sites. One site was positioned within the p6 region (upstream of the PTAP (SEQ ID NO:1) motif) and the other was positioned downstream of the p6 region. Therefore, to generate the desired p6 mutant, GagA7 was cleaved with Bpu10I, agarose gel purified and circularized by re-ligation. E. coli (DH5α) bacterial cells were transformed with the ligated product and streaked onto ampicillin (AMP)-containing LB agar culture plates. Following a 16 hr incubation at 37° C. (with shaking), several colonies were selected and grown in 5 ml of LB-AMP media. Following plasmid purification, a deletion of the p6 region was verified by restriction enzyme digestion (using Bsa I) and by sequence analysis.

### Example 3

### HIV-1 Gag P6 Mutant Vaccine Elicits CMI Response in Mice

Five to seven week old female Balb/c mice were injected intramuscularly with 50 μg of DNA encoding either HIV-1 Gag (GagA7) or p6 mutant Gag. Some of the mice were also administered a Gag p42 protein boost. Five mice per test group received one injection every 2 weeks for a total of 3 DNA injections (2 animals were designated “naïve” animals and did not receive any vaccination). 2 weeks after the final injection, mice were sacrificed, spleens were harvested and splenocytes were prepared and analyzed by ICC, lymphocyte proliferation and CTL activity assays. Mice in the DNA plus protein boost group were given 30 ug of Gag p42 protein boost at weeks 5 and7 and splenocytes were harvested at week 9.

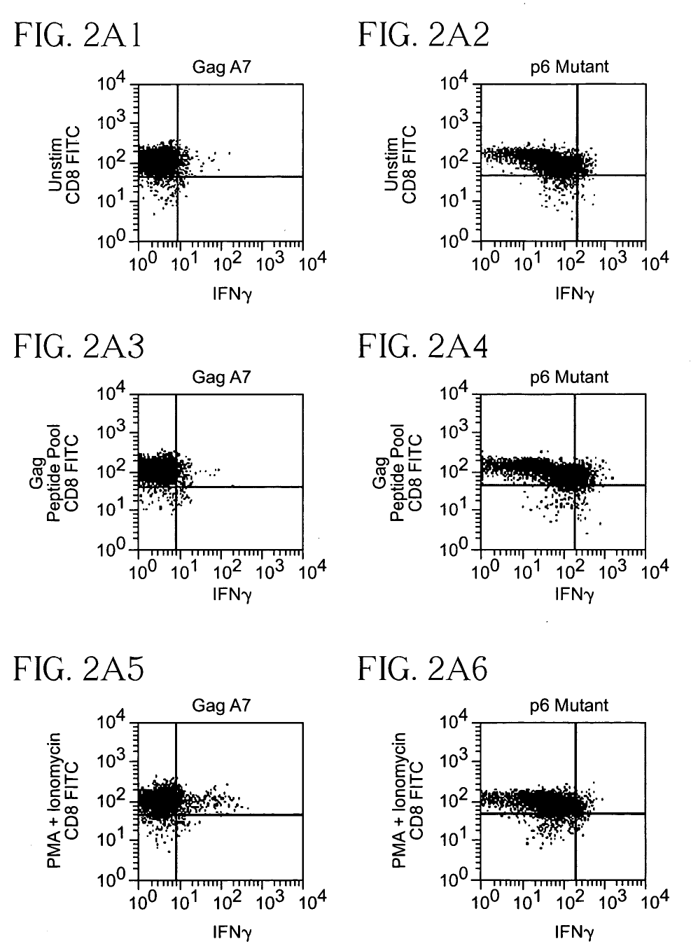

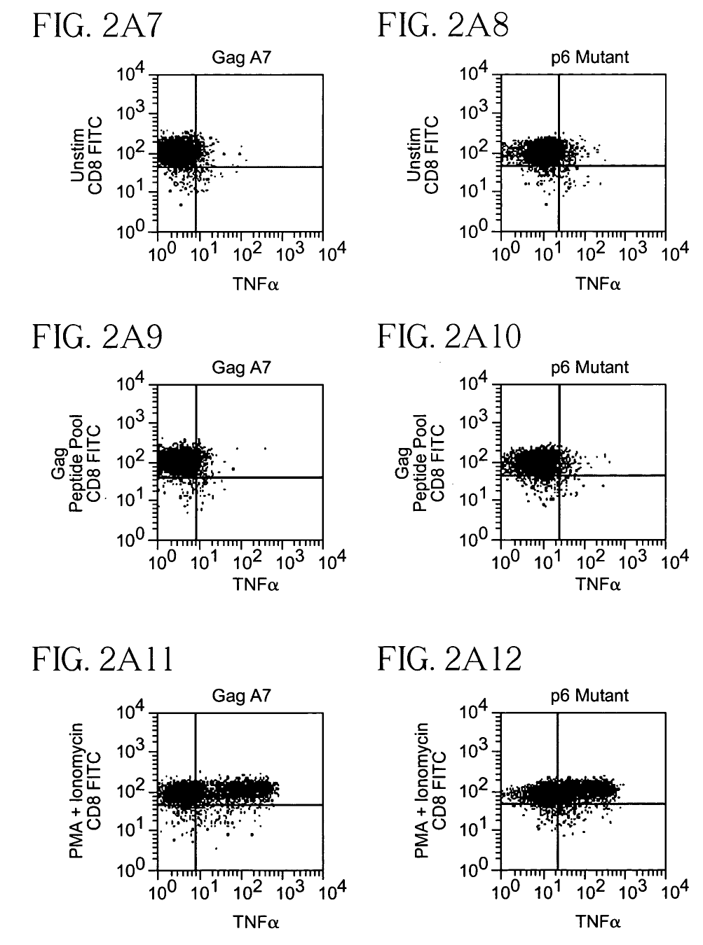

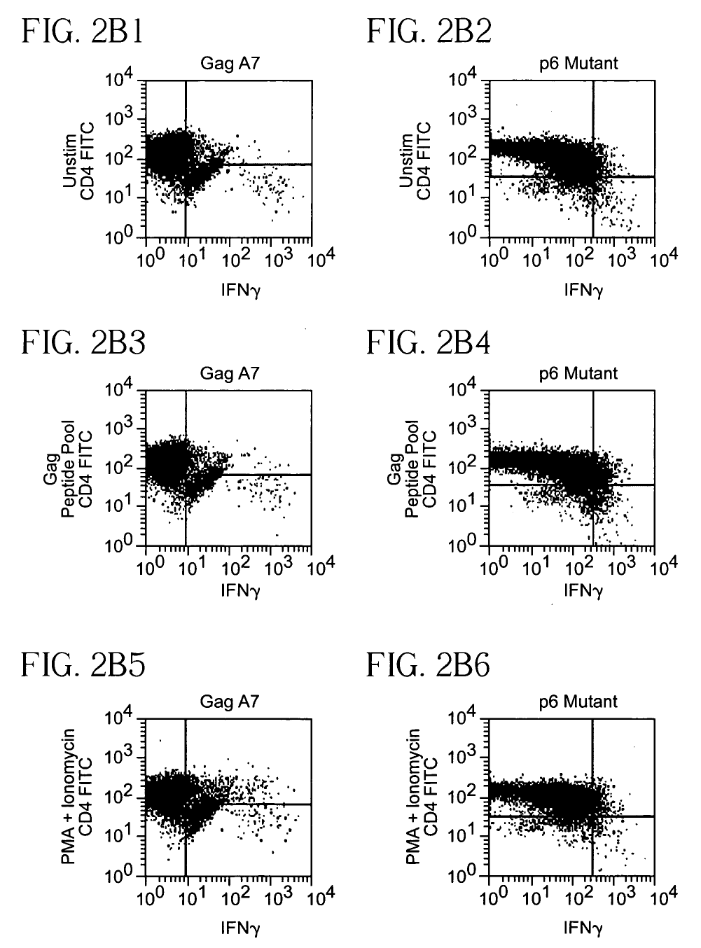

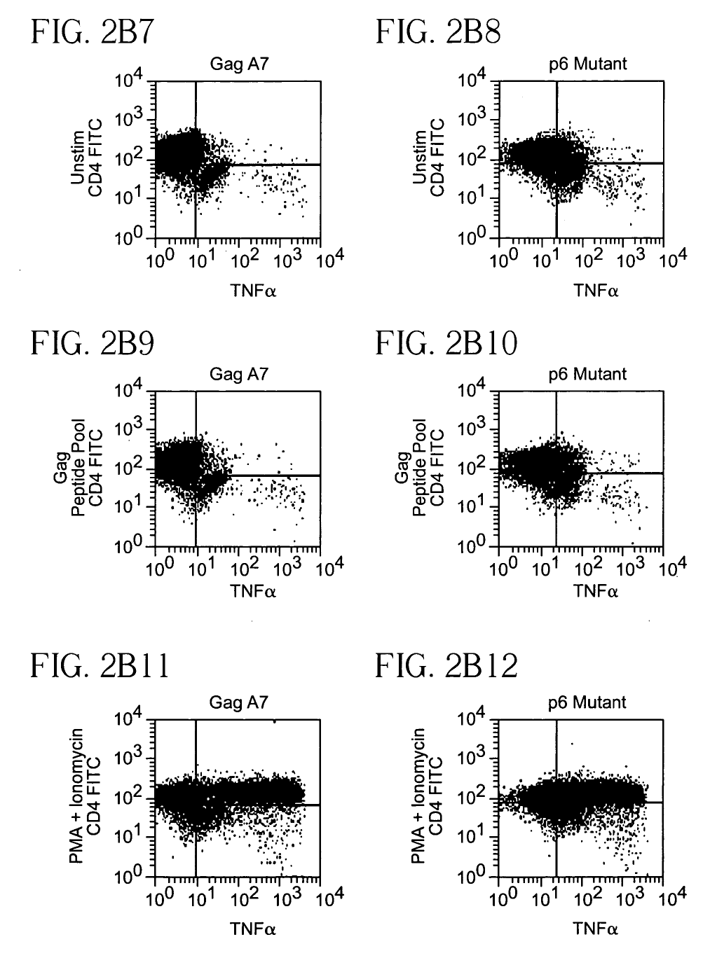

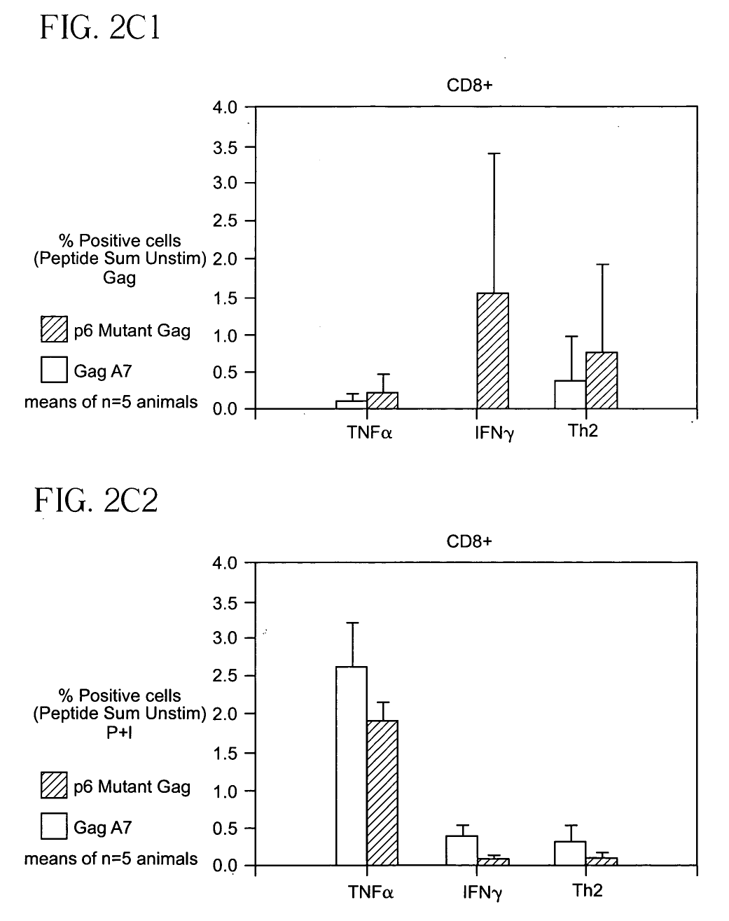

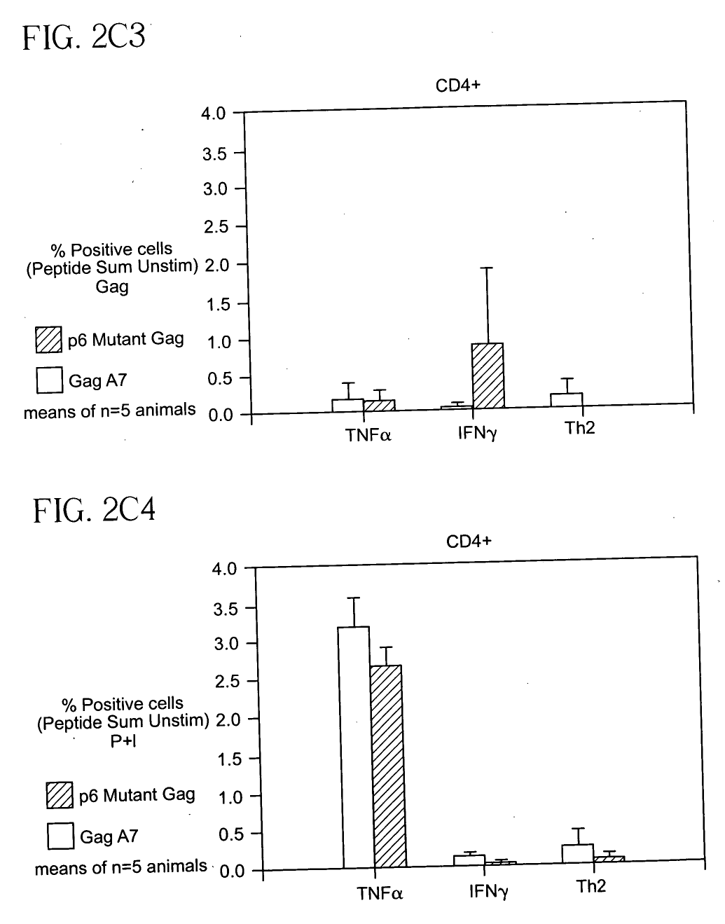

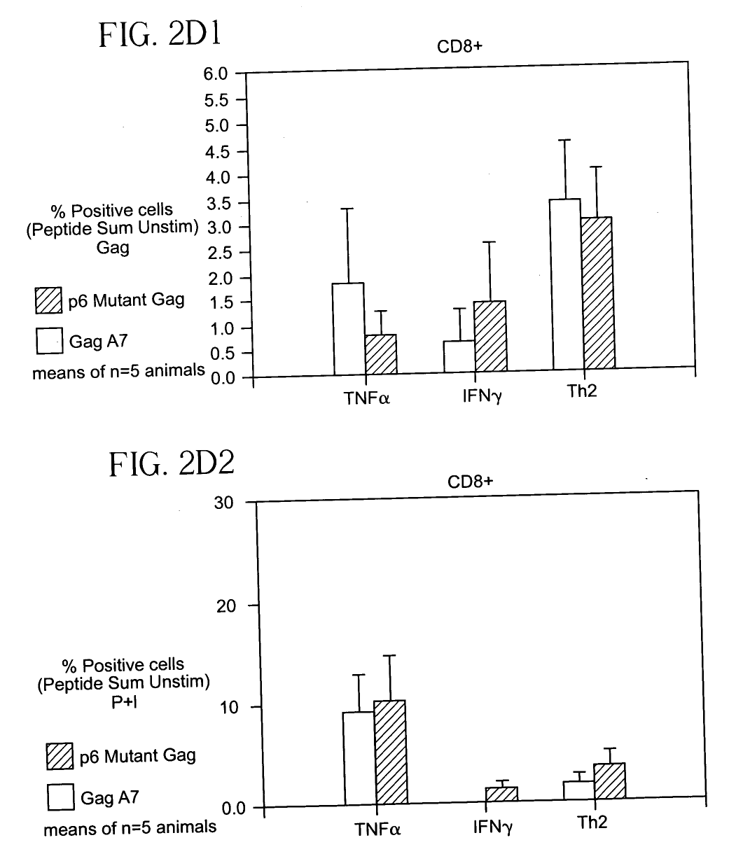

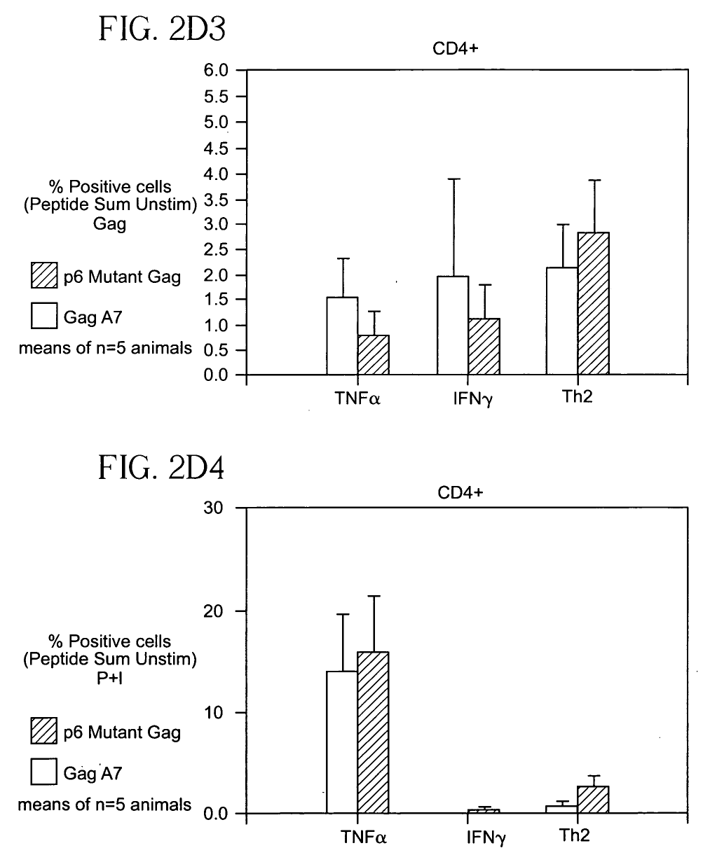

Intracellular Cytokine Staining (ICC): The levels of Th1 (IFNγ, TNFα) and Th2 (IL4, IL5 and IL10) cytokines in CD8+ and CD4+ T cells were assayed by ICC following a 5 hr stimulation with either Gag peptide pool or PMA plus lonomycin (lono). FIGS. 2A and 2B illustrate dot blot analyses (1 representative animal from each test group) of CD8+ T cells producing IFNγ (FIG. 2A, upper), TNFα (FIG. 2A, lower) and of CD4+ T cells producing IFNγ (FIG. 2B, upper), TNFα (FIG. 2B, lower). A summary of the results from one preliminary experiment noting the mean levels of Th1 and Th2 cytokines relative to unstimulated controls are graphically represented in FIG. 2C. As expected, Th1 and Th2 cytokine levels following PMA plus lono stimulation did not differ significantly in either CD8+ or CD4+ T cells when comparing cells isolated from GagA7- or p6 mutant-vaccinated animals. It was found that in T cells stimulated with Gag peptide pool, IFNγ levels were augmented in both CD8+ and CD4+ T cells isolated from p6 mutant-vaccinees relative to GagA7. Neither TNFα nor Th2 cytokine levels in CD8+ and CD4+ T cells differed substantially among the two test groups. While PMA plus lono stimulation produced comparable Th1 and Th2 cytokine levels in naïve animals as compared to that of vaccinated test animals, there was no IFNγ, TNFα or Th2 cytokines produced in response to Gag peptide pool-stimulation (data not shown). Intriguingly, IFNγ, TNFα and Th2 (IL4, IL5, IL10) cytokine levels were found to be greater in p6 mutant-vaccinated mice relative to Gag A7, independent of the stimulation condition used. Following Gag p42 protein boost, Th1 cytokine production was also found to be greater in p6 mutant Gag vaccinated mice as compared to Gag A7 mice (FIG. 2D). However, this increase was not as dramatic as that noted in DNA-only immunized mice (FIG. 2C).

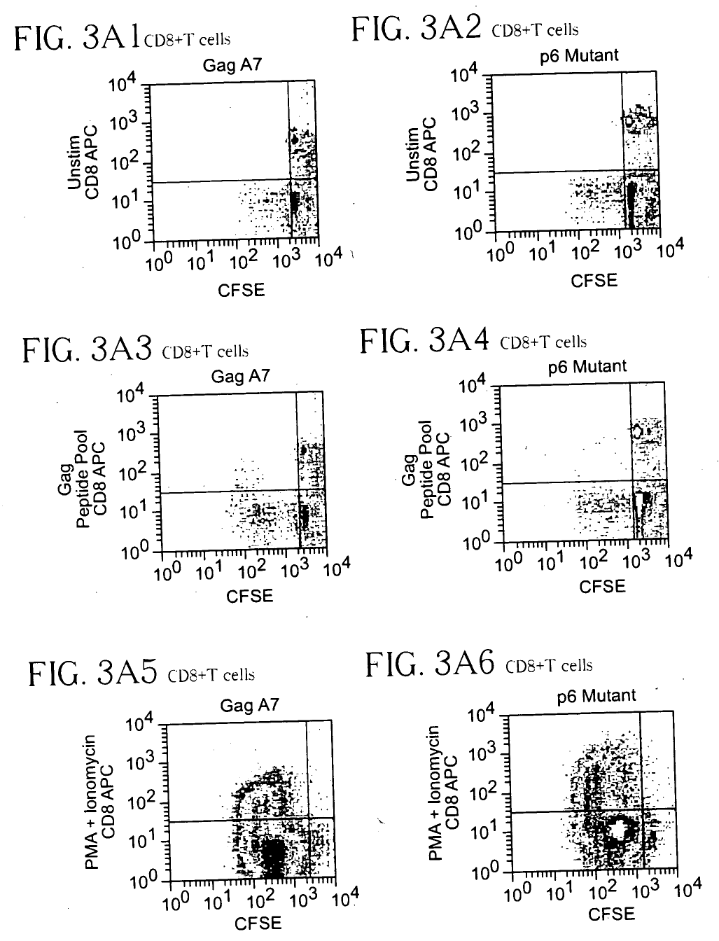

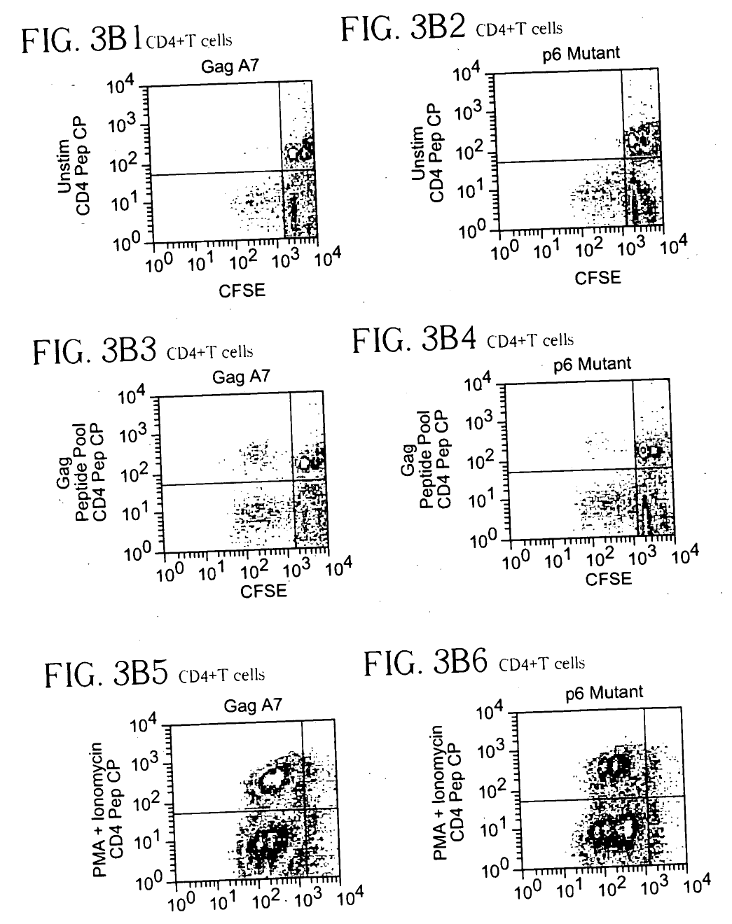

Lymphocyte Proliferation: Following a 72 hour stimulation of splenocytes with Gag peptide pool or PMA plus Iono, proliferation of T cells, isolated from GagA7- and p6 mutant-vaccinated animals, was assayed using CFSE detection. FIGS. 3A and 3B illustrate a density blot analyses of CD8+ (FIG. 3A) and CD4+ (FIG. 3B) T cells from representative animals of the 2 test groups. There was no significant difference in the level of proliferation between the 2 test groups (FIG. 3B and data not shown). A similar result was also found in DNA plus protein vaccinated mice. The preliminary results suggest that deletion of the HIV-1 Gag P6 region does not negatively alter the level of proliferation in T cells.

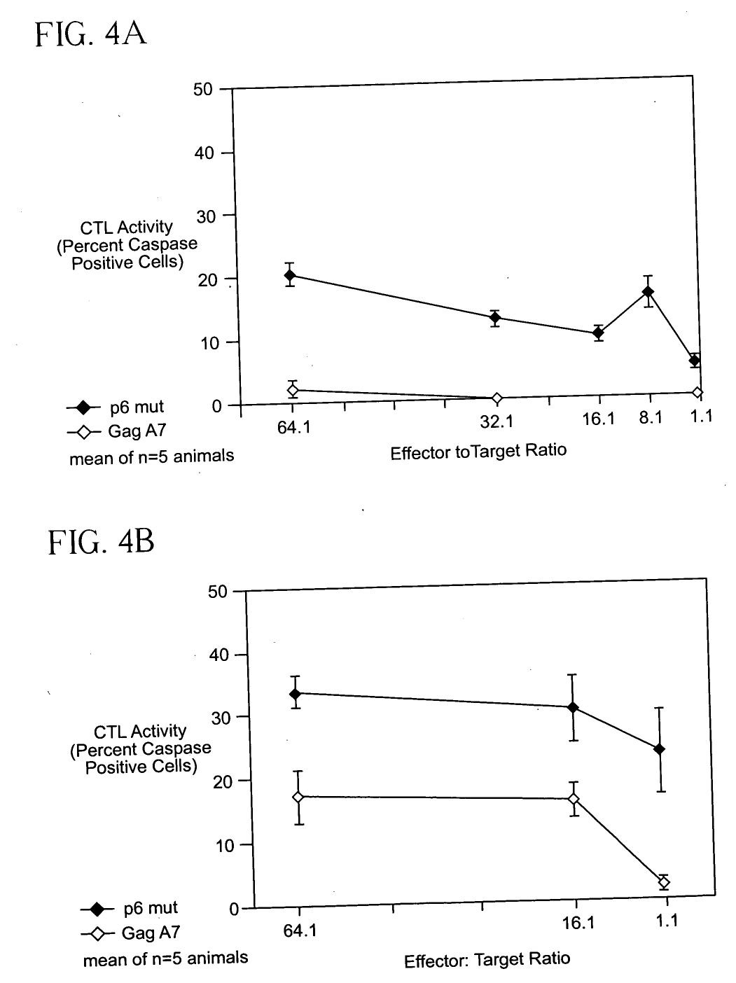

CTL Activity: CTL activity was assayed in effector splenocytes isolated from the 2 vaccinated test groups using the FCC assay. The results are shown in FIG. 4A for DNA vaccinated mice and the results are shown in FIG. 4B for DNA plus protein vaccinated mice. The results show that mice vaccinated with the p6 mutant vaccine (FIGS. 4A, 4B closed circles) display a higher CTL activity as compared to those vaccinated with the GagA7 vaccine (FIGS. 4A, 4B open circles).

In summary, the data shows that an HIV-1 Gag PTAP (SEQ ID NO:1) mutant vaccine elicits an increased CMI response as noted by increased Th1 cytokine production and increased CTL activity.

### Example 4

### Preparing Additional HIV-1 Gag P6 Mutants

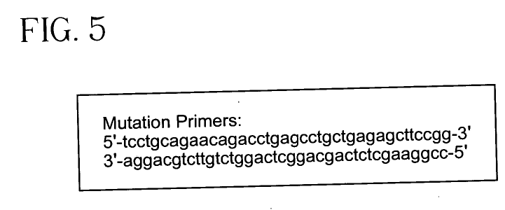

Additional HIV-1 Gag p6 PTAP (SEQ ID NO:1) deletion mutants are prepared using the QuikChange Site-Directed Mutagenesis protocol (Stratagene). Briefly, this protocol utilizes a supercoiled double-stranded DNA template, PfuTurbo DNA polymerase, and two synthetic oligonucleotide primers that contain the desired mutation that are complementary to opposite stands of the target DNA. The primers are extended during temperature cycling which incorporates the desired mutation resulting in a mutant product containing staggered nicks. DpnI, an endonuclease specific for methylated and hemimethylated DNA, is subsequently used after the thermocycling reaction to digest the parental DNA template; thus, allowing isolation of the mutated plasmid. Candidate mutant clones that grow successfully in transformed Epicurean Coli XL-1 Blue supercompetent cells are prepared using a standard plasmid purification protocol and are sequenced to verify the mutation. These mutations are prepared using mammalian expression plasmids expressing HIV-1 (i) Gag alone (FIG. 5), (ii) Gag and Pol, (iii) Gag, Pol and Env and (iv) the HXB2 molecular clone (Accession No. K03455).

### Example 5

### Preparing SIV-1 Gag p6 Mutants

The same methodology used in Example 4 to generate the HIV-1 Gag PTAP (SEQ ID NO:1) deletion mutants is used to produce SIV Gag PTAP (SEQ ID NO:1) mutants. The QuikChange Mutagenesis protocol is used to carry out mutations using mammalian expression plasmids expressing SIV (i) Gag alone, (ii) Gag and Pol, (iii) Gag, Pol and Env and (iv) the SIV251 molecular clone (Accession No. M19499).

### Example 6

### Effects of Gag PTAP ( SEQ ID NO:1) Mutant Overexpression on Viral Particle Budding and Intracellular Presence

The PTAP (SEQ ID NO:1) mutant plasmids prepared and sequence as described in Examples 4 and 5 and the wild-type plasmids are transiently transfected into 293T cells using the CalPhos protocol (Clontech). Briefly, the plasmid DNA is added to a calcium phosphate solution, premixed with an equal volume of hepes-buffered saline and incubated at room temperature for 20 minutes. The entire solution is then added to a subconfluent cell culture of 293T cells and incubated for 12 hr at 37° C. The tansfection solution is then replaced with complete growth media and incubated for an additional 12 hr at 37° C. Following transient transfection, the viral particles that are expressed in 293 cells are analyzed by scanning and transmission electron microscopy. This analysis is used to examine the effects of PTAP mutation on (i) the ability of viral particles to bud as well as (ii) the intracellular retention of HIV-1 Gag or SIV Gag.

### Example 7

### Evaluation of the CMI Response Using the Gag PTAP (SEO ID NO:1) Mutants in a Murine Model System

The CMI responses are evaluated in mice using the HIV-1 Gag PTAP (SEQ ID NO:1) mutant vaccines or the SIV Gag PTAP (SEQ ID NO:1) mutant vaccines. Briefly, 5-7 week old female Balb/c mice are vaccinated intramuscularly with a mammalian expression plasmid encoding Gag (HIV-1 or SIV) or with the PTAP (SEQ ID NO:1) deletion mutant plasmids (HIV-1 or SIV) prepared in Examples 4 or 5. Following 3 DNA vaccinations (administered at 2 week intervals), the animals are sacrificed and splenocytes are tested for CMI responses using the assays described in Example 1 to assess cytokine production, lymphocyte proliferation and CTL activity. Following a 5 hour stimulation of splenocytes with Gag peptide pool, ICC is used to evaluate Th1 (IFNγ, TNFα) and Th2 (IL4, IL5, IL10) cytokine production in CD8+ and CD4+ T cells. IFNγ production and secretion by splenocytes is also measured using an ELISPOT assay following an 18 hour cell stimulation. Proliferation of CD8+ and CD4+ T lymphocytes is assayed by CFSE labelling and detection by flow cytometry. Lastly, the FCC assay is employed to evaluate the CTL activity of splenocytes stimulated ex vivo with Gag peptide pools. After evaluating the effects of the PTAP (SEQ ID NO:1) mutation on CMI response using 3 DNA vaccinations, these responses using a DNA prime plus protein boost strategy or other (i.e., live or attenuated virus) boost approach is tested. This follow-up study tests whether the CMI responses (augmented using the PTAP (SEQ ID NO:1) mutant vaccine) are further augmented with a boost regimen. The most promising candidates on the basis of these tests are used for subsequent testing in nonhuman primates.

### Example 8

### Evaluation of the CMI Response Using the Gag PTAP (SEO ID NO:1) Mutants in a Nonhuman Primate Model System

The CMI responses are evaluated in nonhuman primates using the SIV Gag PTAP (SEQ ID NO:1) mutant vaccines. Briefly, cynomolgous macaque are vaccinated with a mammalian expression plasmid encoding SIV Gag or with the PTAP (SEQ ID NO:1) deletion mutant plasmids prepared in Example 5. Following 3 DNA vaccinations (administered at 2 week intervals), peripheral blood mononuclear cells (PBMCs) areisolated from the animals and the CMI responses are measured following Gag peptide pool stimulation presented in the context of EBV-transforned autologous B lymphocytes. Following a 5 hour stimulation of cynomolgous macaque PBMCs with Gag peptide pools, ICC is employed to evaluate Th1 (IFNγ, TNFα) and Th2 (IL4, IL10) cytokine production in CD8+ and CD4+ T cells. IFNγ production and secretion by stimulated PBMCs is also measured using an ELISPOT assay after an 18 hour stimulation. Proliferation of CD8+ and CD4+ T cells is assayed using CFSE, and the CTL activity is assessed using the FCC assay. As with the murine vaccination studies, this nonhuman primate study includes a DNA prime plus protein boost approach or other (i.e., live or attenuated virus) boost strategy. This follow-up study tests whether the CMI responses (augmented using the PTAP (SEQ ID NO:1) mutant vaccine) are further augmented with a boost regiment.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### BIBLIOGRAPHY

1. Harrer, E., Harrer, T., Buchbinder, S., Mann, D. L., Feinberg, M., Yilma, T., Johnson, R. P., and Walker, B. D. (1994). AIDS Res Hum Retroviruses 10 Suppl 2:S77-78.
2. Koup, R. A., Safrit, J. T., Cao, Y., Andrews, C. A., McLeod, G., Borkowsky, W., Farthing, C., and Ho, D. D. (1994). J Virol 68:4650-4655.
3. Borrow, P., Lewicki, H., Hahn, B. H., Shaw, G. M., and Oldstone, M. B. (1994). J Virol 68:6103-6110.
4. Klenerman, P., Phillips, R. E., Rinaldo, C. R., Wahl, L. M., Ogg, G., May, R. M., McMichael, A. J., and Nowak, M. A. (1996). Proc Natl Acad Sci USA 93:15323-15328.
5. Wagner, R., Shao, Y., and Wolf, H. (1999). Vaccine 17:1706-1710.
6. Yu, X. G., Addo, M. M., Rosenberg, E. S., Rodriguez, W. R., Lee, P. K., Fitzpatrick, C. A., Johnston, M. N., Strick, D., Goulder, P. J., Walker, B. D., and Altfeld, M. (2002). J Virol 76:8690-8701.
7. Gruters, R. A., van Baalen, C. A., and Osterhaus, A. D. (2002). Vaccine 20:2011-2015.
8. Rasmussen, R. A., Hofmann-Lehman, R., Montefiori, D. C., Li, P. L., Liska, V., Vlasak, J., Baba, T. W., Schmitz, J. E., Kuroda, M. J., Robinson, H. L., McClure, H. M., Lu, S., Hu, S. L., Rizvi, T. A., and Ruprecht, R. M. (2002). J Med Primatol 31:40-60.
9. Newman, M. J. (2002) Curr Opin Investig Drugs 3, 374-378.
10. Hanke, T., McMichael, A. J., Mwau, M., Wee, E. G., Ceberej, I., Patel, S., Sutton, J., Tomlinson, M., and Samuel, R. V. (2002). Vaccine 20:1995-1998.
11. Hanke, T. (2003). Curr Opin Mol Ther 5:25-32.
12. Allen, T. M., Vogel, T. U., Fuller, D. H., Mothe, B. R., Steffen, S., Boyson, J. E., Shipley, T., Fuller, J., Hanke, T., Sette, A., Altman, J. D., Moss, B., McMichael, A. J., and Watkins, D. I. (2000). J Immunol 164:4968-4978.
13. Caulfield, M. J., Wang, S., Smith, J. G., Tobery, T. W., Liu, X., Davies, M. E., Casimiro, D. R., Fu, T. M., Simon, A., Evans, R. K., Emini, E. A., and Shiver, J. (2002). J Virol 76:10038-10043.
14. Ott, D. E., Coren, L. V., Copeland, T. D., Kane, B. P., Johnson, D. G., Sowder, R. C., 2nd, Yoshinaka, Y., Oroszlan, S., Arthur, L. O., and Henderson, L. E. (1998). J Virol 72:2962-2968.
15. Ott, D. E., Coren, L. V., Chertova, E. N., Gagliardi, T. D., and Schubert, U. (2000). Virology 278:111-121.
16. Patnaik, A., Chau, V., and Wills, J. W. (2000). Proc Natl Acad Sci USA 97:13069-13074.
17. Schubert, U., Ott, D. E., Chertova, E. N., Welker, R., Tessmer, U., Princiotta, M. F., Bennink, J. R., Krausslich, H. G., and Yewdell, J. W. (2000). Proc Natl Acad Sci USA 97:13057-13062.
18. Gottlinger, H. G., Dorfman, T., Sodroski, J. G., and Haseltine, W. A. (1991). Proc Natl Acad Sci USA 88:3195-3199.
19. Xiang, Y., Cameron, C. E., Wills, J. W., and Leis, J. (1996). J Virol 70:5695-5700.
20. Puffer, B. A., Parent, L. J., Wills, J. W., and Montelaro, R. C. (1997). J Virol 71:6541-6546.
21. Huang, M., Orenstein, J. M., Martin, M. A., and Freed, E. O. (1995). J Virol 69:6810-6818.
22. Li, F., Chen, C., Puffer, B. A., and Montelaro, R. C. (2002). J Virol 76:1569-1577.
23. Yuan, B., Campbell, S., Bacharach, E., Rein, A., and Goff, S. P. (2000). J Virol 74:7250-7260.
24. Yasuda, J., and Hunter, E. (1998). J Virol 72:4095-4103.
25. Garrus, J. E., von Schwedler, U. K., Pornillos, O. W., Morham, S. G., Zavitz, K. H., Wang, H. E., Wettstein, D. A., Stray, K. M., Cote, M., Rich, R. L., Myszka, D. G., and Sundquist, W. I. (2001). Cell 107:55-65.
26. Demirov, D. G., Ono, A., Orenstein, J. M., and Freed, E. O. (2002) Proc Natl Acad Sci USA 99:955-960.
27. Cristillo, A. D., Macri, M. J., and Bierer, B. E. (2003). J. Biol. Chem. 278(36):34587-34597.
28. Cristillo, A. D. and Bierer, B. E. (2002). J. Biol. Chem. 277(6):4465-4476.
29. Cristillo, A. D., Macri, M. J., and Bierer, B. E. Cytokine regulation by calcineurin and by T cell coreceptor signaling. Journal of Clinical Immunology, Submitted
30. Cristillo, A. D., Macri, M. J., and Bierer, B. E. (2003). Blood 101(1):216-25.
31. O'Shea J J, M. A., Lipsky P. (2002). Nature Rev Immunol. 2:37-45.
32. Liu, L., Chahroudi, A., Silvestri, G., Wernett, M. E., Kaiser, W. J., Safrit, J. T., Komoriya, A., Altman, J. D., Packard, B. Z., and Feinberg, M. B. (2002). Nat Med 8:185-189.
33. Tang, D. C., De Vit, M., and Johnston, S. A. (1992). Nature 356(6365):152-4.
34. Ulmer, J. B., Donnelly, J. J., Parker, S. E., Rhodes, G. H., Felgner, P. L., Dwarki, V. J., Gromkowski, S. H., Deck, R. R., De Witt, C. M., Friedman, A., and et al. (1993). Science 259(5102):1745-9.
35. Fynan, E. F., Webster, R. G., Fuller, D. H., Haynes, J. R., Santoro, J. C., and Robinson, H. L. (1993). Proc Natl Acad Sci USA 90(24):11478-82.
36. Robinson, H. L., Hunt, L. A., and Webster, R. G. (1993). Vaccine 11(9):957-60.
37. Xiang, Z., and Ertl, H. C. (1995). Immunity 2(2):129-35.
38. Barry, M. A., Lai, W. C., and Johnston, S. A. (1995). Nature 377(6550):632-5.
39. Feltquate, D. M., Heaney, S., Webster, R. G., and Robinson, H. L. (1997). Journal of Immunology 158(5):2278-84.
40. Robinson, H. L., and Pertmer, T. M. (1998). Nucleic Acid Immunizations. In “Current Protocols in Immunology” (R. Coico, Ed.), Vol. 1, pp. 2.14.1-2.14.19. 3 vols. John Wiley & Sons, Inc., New York.
41. Bohm, W., Kuhrober, A., Paier, T., Mertens, T., Reimann, J., and Schirmbeck, R. (1996). J Immunol Methods 193(1):29-40.
42. Chapman B S, Thayer R M, Vincent K A, Haigwood N L. (1991). Nucleic Acids Research 19(14):3979-86.
43. Hartikka, J., Sawdey, M., Comefert-Jensen, F., Margalith, M., Barnhart, K., Nolasco, M., Vahlsing, H. L., Meek, J., Marquet, M., Hobart, P., Norman, J., and Manthorpe, M. (1996). Hum Gene Ther 7(10):1205-17.
44. Manthorpe, M., Comefert-Jensen, F., Hartikka, J., Feigner, J., Rundell, A., Margalith, M., and Dwarki, V. (1993). Hum Gene Ther 4(4):419-31.
45. Montgomery, D. L., Shiver, J. W., Leander, K. R., Perry, H. C., Friedman, A., Martinez, D., Ulmer, J. B., Donnelly, J. J., and Liu, M. A. (1993). DNA Cell Biol 12(9):777-83.
46. Andre, S., Seed, B., Eberle, J., Schraut, W., Bultmann, A., and Haas, J. (1998). J Virol 72(2):1497-503.
47. Uchijima, M., Yoshida, A., Nagata, T., and Koide, Y. (1998). J Immunol 161(10):5594-9.
48. Iwasaki, A., Stiemholm, B. J., Chan, A. K., Berinstein, N. L., and Barber, B. H. (1997). J Immunol 158(10):4591-601.
49. Wild, J., Gruner, B., Metzger, K., Kuhrober, A., Pudollek, H. P., Hauser, H., Schirmbeck, R., and Reimann, J. (1998). Vaccine 16(4):353-60.
50. Pertmer, T. M., Roberts, T. R., and Haynes, J. R. (1996). J Virol 70(9):6119-25.
51. Kawabata, K., Takakura, Y., and Hashida, M. (1995). Pharm Res 12(6):825-30.
52. Lew, D., Parker, S. E., Latimer, T., Abai, A. M., Kuwahara-Rundell, A., Doh, S. G., Yang, Z. Y., Laface, D., Gromkowski, S. H., and Nabel, G. J., et al. (1995). Cancer gene therapy using plasmid DNA: Pharmakinetic study of DNA following injection in mice [see comments]. Hum. Gene Ther. 6:553.
53. Bohm, W., Mertens, T., Schirmbeck, R., and Reimann, J. (1998). Vaccine 16(9-10):949-54.
54. Asakura, Y., Hinkula, J., Leandersson, A. C., Fukushima, J., Okuda, K., and Wahren, B. (1997). Scand J Immunol46(4):326-30.
55. Sasaki, S., Hamajima, K., Fukushima, J., Ihata, A., Ishii, N., Gorai, I., Hirahara, F., Mohri, H., and Okuda, K. (1998). Infect Immun 66(2):823-6.
56. Livingston, J. B., Lu, S., Robinson, H. L., and Anderson, D. J. (1995). Annals of the New York Academy of Sciences 772:265-7.
57. McCluskie, M. J., Chu, Y., Xia, J. L., Jessee, J., Gebyehu, G., and Davis, H. L. (1998). Antisense Nucleic Acid Drug Dev 8(5):401-14.
58. Chen, S. C., Jones, D. H., Fynan, E. F., Farrar, G. H., Clegg, J. C., Greenberg, H. B., and Herrmann, J. E. (1998). J Virol 72(7):5757-61.
59. Jones, D. H., Corris, S., McDonald, S., Clegg, J. C., and Farrar, G. H. (1997). Vaccine 15(8):814-7.
60. Sizemore, D. R., Branstrorn, A. A., and Sadoff, J. C. (1995). Science 270(5234):299-302.
61. Sizemore, D. R., Branstrom, A. A., and Sadoff, J. C. (1997). Vaccine 15(8):804-7.
62. Richmond, J. F., Lu, S., Santoro, J. C., Weng, J., Hu, S. L., Montefiori, D. C., and Robinson, H. L. (1998). J Virol 72(11):9092-9 100.
63. Letvin, N. L., Montefiori, D. C., Yasutomi, Y., Perry, H. C., Davies, M. E., Lekutis, C., Alroy, M., Freed, D. C., Lord, C. I., Handt, L. K., Liu, M. A., and Shiver, J. W. (1997). Proc Natl Acad Sci USA 94(17):9378-83.
64. Hanke, T., Blanchard, T. J., Schneider, J., Hannan, C. M., Becker, M., Gilbert S. C., Hill, A. V., Smith, G. L., and McMichael, A. (1998). Vaccine 16(5):439-45.
65. Kent S. J., Zhao, A., Best, S. J., Chandler, J. D., Boyle, D. B., and Ramshaw, 1. A. (1998). J Virol 72(12):10180-8.
66. Schneider, J., Gilbert, S. C., Blanchard, T. J., Hanke, T., Robson, K. J., Hannan, C. M., Becker, M., Sinden, R_Smith, G. L., and Hill, A. V. (1998). Nat Med 4(4):397-402.
67. Tang, D.-C., DeVit, M., Johnston, S. A. (1992). Nature 356:152-154.
68. Barry, M. A., Lai, W. C., Johnston, S. A. (1995). Nature 377:632-635.
69. Cohen, J. 19939). Science 262:1820-1821.
70. Berman, P. W., Gregory, T. J., Riddle, L., Nakamura, G. R., Champe, M. A., Porter, J. P., Wurm, F. M., Hirshberg, R. D., Cobb, E. K., and Eichberg, J. W. (1990). Nature 345:622-625.
71. El-Amad, Z., Murthy, K. K., Higgins. K., Cobb, E. K., Haigwood, N. K., Levy, J. A., and Steimer, K. S. (1995). AIDS 9:1313-1322.
72. Emini, E. A., Nara, P. L., Schlief, W. A., Lewis, J. A., Davide, J. P., Lee, D. R., Kessler, J., Conley, S., Matshushita, S., Putney, S. D., Gerety, R. J., and Eichberg, J. W. (1990). J Virol. 64:3674-3678.
73. Emini, E. A., Schleif, W. A., Nunberg, J. H., Conley, A. J., Eda, Y., Tokiyoshi, S., Putney, S. D., Matsushita, S., Cobb, K. E., Jett, C. M., Eichberg, J. W., and Murthy, K. K. (1992). Nature 355:728-730.
74. Girard, M., Kieny, M.-P., Pinter, A., Barre-Sinoussi, F., Nara, P., Kolbe, H., Kusumi, K., Chaput, A., Reinhart, T., Muchmore, E., Ronco, J., Kaczorek, M., Gomard, E., Gluckman, J.-C., and Fultz, P. N. (1991). Proc. Natl. Acad. Sci. USA 88:542-546.
75. Shibata, R., Siemon, C., Cho, M. W., Arthur, L. O., Nigida, S.M., Jr., Matthews, T., Sawyer, L. A., Schultz, A., Murthy, K. K., Israel, Z., Javadian, A., Frost, P., Kennedy, R. C., Lane, H. C., and Martin, M. A. (1996). J. Virol. 70:4361-4369.
76. Goeddel, D. V. (1990). “Gene Expression Technology.” In Methods in Enzymology 185, Academic Press, San Diego, Calif.
77. Calame, K. and Eaton, S. (1988). Adv. Immunol. 43:235-275.
78. Winoto, A. and Baltimore, D. (1989). EMBO J. 8:729-733.
79. Banerji, J., Olson, L. and Schaffner, W. (1983). Cell 33:729-740.
80. Queen, C. and Baltimore, D. (1983). Cell 33:741-748.
81. Freed, E. O. (1998). Virology 251:1-15.
82. Harris, E. L. V. and Angal, S. eds. (1990). Protein Purification Applications: A Practical Approach, IRL Press, Oxford.
83. Kirnbauer, R., Taub, J., Greenstone, H., Roden, R., Durst, M., Gissmann, L., Lowy, D. R., and Schiller, J. T. (1993). J. Virol. 67:6929-6936.
84. Freed, E. O. (2002). J. Virol. 76:4679-4687.

1. A DNA molecule comprising a nucleic acid comprising a deletion mutation of the budding mediating motif of a viral protein encoded by the nucleic acid, wherein the viral protein is associated with the virus budding process.

2. The DNA molecule of claim 1, wherein the budding mediating motif comprises an amino acid sequence selected from the group consisting of PTAP (SEQ ID NO:1), PPXY (SEQ ID NO:2), YXXL (SEQ ID NO:3) and a combination thereof.

3. The DNA molecule of claim 2, wherein the viral protein is a Gag protein of a retrovirus or a matrix protein of a rhabdovirus or filovirus.

4. The DNA molecule of claim 1, wherein the viral protein is a Gag protein of a retrovirus or a matrix protein of a rhabdovirus or filovirus.

5. The DNA molecule of claim 1, wherein at least one codon for the budding mediating motif is deleted.

6. The DNA molecule of claim 5, wherein one or more codons surrounding the budding mediating motif are deleted.

7. The DNA molecule of claim 1 which further comprises one or more additional nucleic acids, each encoding an additional viral protein.

8. The DNA molecule of claim 7, wherein the additional viral proteins are selected from the group consisting of HIV-1 Pol, Env, Rev, Tat and Nef.

9. The DNA molecule of claim 7 which comprises a molecular clone of HIV-1 or SIV.

10. A vector comprising the DNA molecule of claim 1.

11. A composition comprising the vector of claim 10.

12. A composition comprising the DNA molecule of claim 1.

13. A method for immunizing a subject which comprises administering an immunizing effective amount of the DNA molecule of claim 1.

14. The method of claim 13 comprising further administering a recombinant protein or vector boost.

15. A method for immunizing a subject which comprises administering an immunizing effective amount of the vector in claim 10.

16. The method of claim 15 comprising further administering a recombinant protein boost.

17. A method for immunizing a subject which comprises administering an immunizing effective amount of the composition of claim 11.

18. The method of claim 17 comprising further administering a recombinant protein boost.

19. A method for immunizing a subject with comprises administering an immunizing effective amount of the composition of claim 12.

20. The method of claim 19 further comprising administering a recombinant protein or vector boost.

21. A method for augmenting a cellular immune response to a virus which comprises administering an effective amount of the DNA molecule of claim 1 to augment the cellular immune response to the virus.

22. The method of claim 21 further comprising administering a recombinant protein or vector boost.

23. A method for augmenting a cellular immune response to a virus which comprises administering an effective amount of the vector of claim 10 to augment the cellular immune response to the virus.

24. The method of claim 23 further comprising administering a recombinant protein boost.
